(19) United States
(12) Patent Application Publication
(10) Pub. No.: US 2010/0041631 A1
(43) Pub. Date: Feb. 18, 2010

(54) METHODS AND AGENTS FOR TREATING TUBERCULOSIS

(75) Inventors: Lindsay D. Eltis, Vancouver (CA); Victor A. Snieckus, Kingston (CA)

Correspondence Address: FENWICK & WEST LLP, SILICON VALLEY CENTER, 801 CALIFORNIA STREET, MOUNTAIN VIEW, CA, 94041, US

(73) Assignees: THE UNIVERSITY OF BRITISH COLUMBIA, Vancouver, BC (CA); QUEEN'S UNIVERSITY AT KINGSTON, Kingston, ON (CA)

(21) Appl. No.: 12/297,433

(22) PCT Filed: Apr. 17, 2007
(86) PCT No.: PCT/CA2007/000648

§ 371 Date: Feb. 26, 2009

Related U.S. Application Data
(60) Provisional application No. 60/792,963, filed on Apr. 19, 2006.

(51) Int. Cl. A61K 31/59; A61K 31/56; C07C 401/00; C12Q 1/26; C12Q 1/34; A61P 31/06

(52) U.S. Cl. 514/167; 514/177; 552/653; 435/25; 435/18

(57) ABSTRACT

The present invention relates to the treatment of tuberculosis (mycobacterial infections) by the use of KshAB complex inhibitors, or a KstD molecule, or a HsaAB complex, or a HsaC molecule, or a HsaD molecule. The application also includes a method for identifying an inhibitor or modulator of the previously mentioned molecules and complexes.

### FIELD OF INVENTION

The present invention relates to mycobacterial infections. More specifically, it relates to therapeutic agents and methods for treating mycobacterial infections.

### BACKGROUND OF THE INVENTION

The genus Mycobacterium includes many pathogens known to cause serious diseases in mammals, including tuberculosis and leprosy. Mycobacterial infections are notoriously difficult to treat. The organisms are hardy due to their unique cell wall, which also confers resistance to a number of antibiotics.

Mycobacteria can be classified into several major groups for purpose of diagnosis and treatment, including M. tuberculosis complex (M. tuberculosis, M. bovis, M. africanum, and M. microti), which cause tuberculosis; M. leprae which causes Hansen's disease or leprosy; M. avium complex (MAC) (M. avium subspecies avium (MAA), subspecies hominis (MAH), and subspecies paratuberculosis (MAP)). MAC, and other nontuberculous mycobacteria (NTM) may cause opportunistic infections in immunocompromised subjects (such as those with HIV) including pulmonary disease resembling tuberculosis, lymphadenitis, skin disease, or disseminated disease.

Tuberculosis (TB) caused by infection with Mycobacterium tuberculosis is a leading cause of mortality from bacterial infection, latently infecting a third of the world's population and killing 2-3 million people each year. After years in decline, M. tuberculosis infections are increasing, largely due to two lethal developments: the association of TB with HIV-infected individuals and the emergence of multidrug-resistant (MDR) strains of M. tuberculosis.

The current standard chemotherapy for TB involves a 6-month treatment program and a cocktail of drugs: an initial 2-month treatment with 4 drugs (isoniazid (INH), rifampin (RIF), pyrazinamide and ethambutol) followed by an additional 4-month treatment with INH and RIF. The inadequacies of this chemotherapy include its toxicity, poor patient compliance with the lengthy treatment, and ineffectiveness against MDR-strains. Chemotherapy against MDR-M. tuberculosis involves more toxic drugs, may last up to 2 years, and is expensive, with the additional complication of even poorer patient compliance.

The mycobacterial cell wall is unique and rich in unusual glycolipids, polysaccharides and lipids such as mycolic acids. Several drugs used in tuberculosis therapy are directed to the synthetic pathways for these lipids, including isoniazid (INH). Recently, arylamine N-acetyltransferase was identified as another cell-wall related target enzyme (BHAKTA et al 2004. J. Exp. Med. 199:1191-1199; ANDERTON et al 2006 Molecular Microbiology 59:181-192). While the genome of M. tuberculosis H37Rv has been fully sequenced and annotated (COLE et al 1998. Nature 393:537-544; CAMUS et al 2002. Microbiology 148:2967-2973), there are many uncharacterized pathways and unidentified genes. 3995 proteins have been predicted, with putative functions assigned to 2058 of these proteins. 376 have no homology to known proteins and may be unique to M. tuberculosis.

Various genes of M. tuberculosis that are involved in cholesterol catabolism are specifically expressed during growth in the macrophage (SCHNAPPINGER et al 2003. J. Exp Med 198:693-704) and a subset of these are essential for survival in the macrophage (RENGARAJAN et al 2005. Proc. Natl. Acad. Sci. USA 102:8327-32).

Several genes have been implicated in preventing acidification of the M. tuberculosis-containing phagosome (PETHE et al 2004. Proc. Natl. Acad. Sci. USA 101:13642-13647).

Macrophage plasma membrane cholesterol has a role in internalization of mycobacteria by macrophages, and sequestration of cholesterol in an in vitro macrophage model inhibits uptake and phagocytosis of mycobacterial. (GATFIELD et al 2000. Science 288:1647-1650; PEYRON et al 2001. J. Immunol. 165:5186-5191).

Catabolic studies in a mycobacterial strain indicate cholesterol is degraded via 4-androstene-3,17-dione (4-AD) with side chain catabolism preceding that of the rings, and side chain degradation at C-17 likely precedes steroid ring degradation (SMITH et al 1993. App. Env. Microbiol. 59:1425-1429).

### SUMMARY OF THE INVENTION

The present invention relates, in part, to enzymes involved in cholesterol degradation in Mycobacterium tuberculosis. Some of these enzymes are essential for growth of M. tuberculosis in the macrophage, and participate in oxygenolytic cleavage of the rings of cholesterol. Described herein are substrate analogues and inhibitors of such enzymes, and methods for synthesizing and assaying such substrate analogues and inhibitors. Such substrate analogues and inhibitors may be used for the treatment of mycobacterial infections, including tuberculosis.

In accordance with one aspect of the invention, there is provided a use of an inhibitor of a KshAB complex, a KstD molecule, a HsaAB complex, a HsaC molecule or a HsaD molecule in the preparation of a medicament for the treatment of tuberculosis in a subject.

In accordance with another aspect of the invention, there is provided a use wherein the KshAB complex molecule comprises a KshA polypeptide comprising an amino acid sequence substantially identical to the amino acid sequence set forth in SEQ ID NO: 17 and a KshB polypeptide comprising an amino acid sequence substantially identical to the amino acid sequence set forth in SEQ ID NO: 19.

In accordance with another aspect of the invention, there is provided a use wherein the inhibitor of the KshAB complex is selected from one or more of the group consisting of 2-chloro-4-androstene-3,17-dione α enantiomer, 2-chloro-4-androstene-3,17-dione β enantiomer, 1-nitro-4-androstene-3,17-dione (1-nitro-4-AD) α enantiomer, 1-nitro-4-androstene-3,17-dione (1-nitro-4-AD)β enantiomer, 4-fluoro-4-androstene-3,17-dione α enantiomer, and 4-fluoro-4-androstene-3,17-dione β enantiomer.

In accordance with another aspect of the invention, there is provided a use wherein the HsaAB complex comprises a HsaA polypeptide comprising an amino acid sequence substantially identical to the amino acid sequence set forth in SEQ ID NO: 21 and a HsaB polypeptide comprising an amino acid sequence substantially identical to the amino acid sequence set forth in SEQ ID NO: 23.

In accordance with another aspect of the invention, there is provided a use wherein the inhibitor of the HsaAB complex is selected from one or more of the group consisting of 2-chloro-3-hydroxy-9,10-seconandrost-1,3,5(10)-triene-9,17-dione, 1-nitro-10-chloro-3-hydroxy-6-methyl-7,8-dihydrostilbene, 4-fluoro-3-hydroxy-9,10-seconandrost-1,3,5(10)-triene-9,17-dione and 7-hydroxymethyl-7-hydroxyethyl-10-Cl-3-hydroxy-6-methyl-7,8-dihydrostilbene.

In accordance with another aspect of the invention, there is provided a use wherein the HsaC molecule comprises a polypeptide comprising an amino acid sequence substantially identical to the amino acid sequence set forth in SEQ ID NO: 25.

In accordance with another aspect of the invention, there is provided a use wherein the inhibitor of the HsaC molecule is selected from one or more of the group consisting of 2-chloro-3,4-dihydroxy-9,10-seconandrost-1,3,5(10)-triene-9,17-dione, 1-nitro-2,3-dihydroxy-6-methyl-7,8-dihydrostilbene, 7,7-dihydroxymethyl 3,4-dihydroxy-9,10-seconandrost-1,3,5(10)-triene-9,17-dione, 7-hydroxymethyl-7-hydroxyethyl-10-Cl-2,3-dihydroxy-6-methyl-7,8-dihydrostilbene, 3-chlorocatechol and 2′,6′-diCl-2,3-dihydroxybiphenyl.

In accordance with another aspect of the invention, there is provided a use wherein the HsaD molecule comprises a polypeptide comprising an amino acid sequence substantially identical to the amino acid sequence set forth in SEQ ID NO: 27.

In accordance with another aspect of the invention, there is provided a use wherein the inhibitor of the HsaD molecule is 3-chloro-4,5-9,10-diseco-3-hydroxy-5,9,17-trioxoandrosta-1(10),2-diene-4-oic acid.

In accordance with another aspect of the invention, there is provided a use wherein the subject is a human.

In accordance with another aspect of the invention, there is provided a use wherein the subject is a guinea pig.

In accordance with another aspect of the invention, there is provided a use wherein the subject is a mouse.

In accordance with another aspect of the invention, there is provided a method of treating tuberculosis in a subject in need thereof by administering to the subject an inhibitor of a KshAB complex molecule, a KstD, a HsaAB complex molecule, a HsaC molecule and a HsaD molecule.

In accordance with another aspect of the invention, there is provided a method wherein the inhibitor of the KshAB complex molecule is selected from one or more of the group consisting of 2-chloro-4-androstene-3,17-dione α enantiomer, 2-chloro-4-androstene-3,17-dione β enantiomer, 1-nitro-4-androstene-3,17-dione (1-nitro-4-AD) α enantiomer, 1-nitro-4-androstene-3,17-dione (1-nitro-4-AD) β enantiomer, 4-fluoro-4-androstene-3,17-dione α enantiomer, and 4-fluoro-4-androstene-3,17-dione β enantiomer.

In accordance with another aspect of the invention, there is provided a method wherein the KshAB complex molecule comprises a KshA polypeptide comprising an amino acid sequence substantially identical to the amino acid sequence set forth in SEQ ID NO: 17 and a KshB polypeptide comprising an amino acid sequence substantially identical to the amino acid sequence set forth in SEQ ID NO: 19.

In accordance with another aspect of the invention, there is provided a method wherein the inhibitor of the HsaAB complex molecule is selected from one or more of the group consisting of 2-chloro-3-hydroxy-9,10-seconandrost-1,3,5(10)-triene-9,17-dione, 1-nitro-10-chloro-3-hydroxy-6-methyl-7,8-dihydrostilbene, 4-fluoro-3-hydroxy-9,10-seconandrost-1,3,5(10)-triene-9,17-dione and 7-hydroxymethyl-7-hydroxyethyl-10-Cl-3-hydroxy-6-methyl-7,8-dihydrostilbene.

In accordance with another aspect of the invention, there is provided a method wherein the HsaAB complex comprises a HsaA polypeptide comprising an amino acid sequence substantially identical to the amino acid sequence set forth in SEQ ID NO: 21 and a HsaB polypeptide comprising an amino acid sequence substantially identical to the amino acid sequence set forth in SEQ ID NO: 23.

In accordance with another aspect of the invention, there is provided a method wherein the inhibitor of the HsaC molecule is selected from one or more of the group consisting of 2-chloro-3,4-dihydroxy-9,10-seconandrost-1,3,5(110)-triene-9,17-dione, 1-nitro-2,3-dihydroxy-6-methyl-7,8-dihydrostilbene, 7,7-dihydroxymethyl 3,4-dihydroxy-9,10-seconandrost-1,3,5(110)-triene-9,17-dione, 7-hydroxymethyl-7-hydroxyethyl-10-Cl-2,3-dihydroxy-6-methyl-7,8-dihydrostilbene, 3-chlorocatechol and 2′,6′-diCl-2,3-dihydroxybiphenyl.

In accordance with another aspect of the invention, there is provided a method wherein the HsaC molecule comprises a polypeptide comprising an amino acid sequence substantially identical to the amino acid sequence set forth in SEQ ID NO: 25.

In accordance with another aspect of the invention, there is provided a method wherein the inhibitor of the HsaD molecule is 3-chloro-4,5-9,10-diseco-3-hydroxy-5,9,17-trioxoandrosta-1(10),2-diene-4-oic acid.

In accordance with another aspect of the invention, there is provided a method wherein the HsaD molecule comprises a polypeptide comprising an amino acid sequence substantially identical to the amino acid sequence set forth in SEQ ID NO: 27.

In accordance with another aspect of the invention, there is provided a method wherein the subject is a human.

In accordance with another aspect of the invention, there is provided a method wherein the subject is a guinea pig.

In accordance with another aspect of the invention, there is provided a method wherein the subject is a mouse.

In accordance with another aspect of the invention, there is provided a compound according to Formula I:

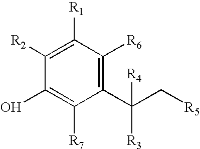

wherein R₁, R₂, R₆ are independently selected from the group consisting of Cl, F, Br, I, —NO₂, —CH═O, —CR═O, —COOH, COOR, CONR₂, COCl, and —CX₃, where X is selected from the group consisting of Cl, Br, F, I, —CN, —SO₃H, —NH₃⁺ and NR₃⁺;
R₇ is any substituent, from one to 10 atoms;
R₃ and R₄ are independently selected from the group consisting of H, —CH₂OH, —CH₂CH₂OH, —CH₂Cl, —CH₂CH₂Cl, —CH₂NH₂, or —CH₂CCl₃; and
R₅ is selected from the group consisting of cycloalkanone, phenyl, purine, pyrimidine and bicycloalkanone.

In accordance with another aspect of the invention, there is provided a compound selected from the group consisting of: 2-chloro-3-hydroxy-9,10-seconandrost-1,3,5(10)-triene-9,17-dione, 1-nitro-10-chloro-3-hydroxy-6-methyl-7,8-dihydrostilbene, 4-fluoro-3-hydroxy-9,10-seconandrost-1,3,5(10)-triene-9,17-dione, and 7-hydroxymethyl-7-hydroxyethyl-10-Cl-3-hydroxy-6-methyl-7,8-dihydrostilbene.

In accordance with another aspect of the invention, there is provided a compound according to Formula II:

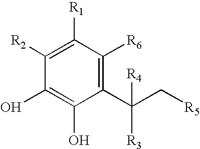

wherein R₁, R₂, R₆ are independently selected from the group consisting of Cl, F, Br, I, —NO₂, —CH═O, —CR═O, —COOH, COOR, CONR₂, COCl, and —CX₃, where X is selected from the group consisting of Cl, Br, F, I, —CN, —SO₃H, —NH₃⁺ and NR₃⁺;
R₃ and R₄ are independently selected from the group consisting of H, —CH₂OH, —CH₂CH₂OH, —CH₂Cl, —CH₂CH₂Cl, —CH₂NH₂, and —CH₂CCl₃; and
R₅ is selected from the group consisting of cycloalkanone, phenyl, purine, pyrimidine, bicycloalkanone and heterocycle.

In accordance with another aspect of the invention, there is provided a compound selected from the group consisting of 2-chloro-3,4-dihydroxy-9,10-seconandrost-1,3,5(10)-triene-9,17-dione, 1-nitro-2,3-dihydroxy-6-methyl-7,8-dihydrostilbene, 7,7-dihydroxymethyl 3,4-dihydroxy-9,10-seconandrost-1,3,5(10)-triene-9,17-dione, 7-hydroxymethyl-7-hydroxyethyl-10-Cl-2,3-dihydroxy-6-methyl-7,8-dihydrostilbene, 3-chlorocatechol and 2′,6′-diCl-2,3-dihydroxybiphenyl.

In accordance with another aspect of the invention, there is provided a compound according to Formula III:

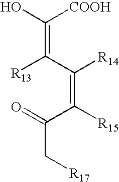

wherein R₁₃, R₁₄ and R₁₅ are independently selected from the group consisting of Cl, F, Br, I, —NO₂, —CH═O, —CR═O, —COOH, COOR, CONR₂, COCl, —CX₃, H, —CH₂OH, —CH₂CH₂OH, —CH₂Cl, —CH₂CH₃, —CH₂CH₂Cl, —CH₂NH₂, or —CH₂CCl₃; where X is a halogen (Cl, Br, F, I), —CN, —SO₃H, —NH₃⁺, NR₃⁺; and
R₁₇ is selected from the group consisting of Cl, F, Br, I, cycloalkanone, phenyl, purine, pyrimidine, bicycloalkanone, heterocycle, cycloalkanone comprising a halogen substituent group, phenyl comprising a halogen substituent group, purine comprising a halogen substituent group, pyrimidine comprising a halogen substituent group, bicycloalkanone comprising a halogen substituent group and heterocycle comprising a halogen substituent group.

In accordance with another aspect of the invention, there is provided a compound selected from the group consisting of 3-chloro-4,5-9,10-diseco-3-hydroxy-5,9,17-trioxoandrosta-1(10),2-diene-4-oic acid (3-Cl-4,9-DSHA) and (2E,4Z)-8-(2-chlorophenyl)-2-hydroxy-4,5-dimethyl-6-oxoocta-2,4-dienoic acid (4,5-diMe-10-Cl-HOPODA).

In accordance with another aspect of the invention, there is provided a pharmaceutical composition comprising a compound according to any one of claims 25 to 30, and a pharmaceutically acceptable carrier.

In accordance with another aspect of the invention, there is provided a pharmaceutical composition further comprising a therapeutic compound for treating tuberculosis.

In accordance with another aspect of the invention, there is provided a pharmaceutical composition wherein the therapeutic compound is selected from one or more of the group consisting of isoniazid (INH), rifampin (RIF), pyrazinamide and ethambutol.

In accordance with another aspect of the invention, there is provided a kit comprising the pharmaceutical composition of any one of claims 31 to 33, together with instructions for treating tuberculosis.

In accordance with another aspect of the invention, there is provided a method for identifying an inhibitor of a KshAB complex molecule, a KstD molecule, a HsaAB complex molecule, a HsaC molecule or a HsaD molecule, the method comprising:

a) providing a test compound;

b) providing a KshAB complex molecule, a KstD molecule, a HsaAB complex molecule, a HsaC molecule or a HsaD molecule;

c) contacting the KshAB complex molecule, KstD molecule, HsaAB complex molecule, HsaC molecule or HsaD molecule with the test compound under conditions suitable for activity of the KshAB complex molecule, KstD molecule, HsaAB complex molecule, HsaC molecule or HsaD molecule; and

d) determining the activity of the KshAB complex molecule, KstD molecule, HsaAB complex molecule, HsaC molecule or HsaD molecule in the presence and absence of the test compound;

wherein the test compound is an inhibitor of the KshAB complex molecule, KstD molecule, HsaAB complex molecule, HsaC molecule or HsaD molecule if the activity of the KshAB complex molecule, KstD molecule, HsaAB complex molecule, HsaC molecule or HsaD molecule is greater in the absence of the test compound compared to the presence of the test compound.

In accordance with another aspect of the invention, there is provided a method for identifying a modulator of a KshAB complex molecule, a KstD molecule, a HsaAB complex molecule, a HsaC molecule or a HsaD molecule, the method comprising:

a) providing a test compound;

b) providing a reference compound;

c) providing a KshAB complex molecule, a KstD molecule, a HsaAB complex molecule, a HsaC molecule or a HsaD molecule;

d) contacting the KshAB complex molecule, KstD molecule, HsaAB complex molecule, HsaC molecule or HsaD molecule with the test compound and with the reference compound under conditions suitable for activity of the KshAB complex molecule, KstD molecule, HsaAB complex molecule, HsaC molecule or HsaD molecule; and

e) determining the activity of the KshAB complex molecule, KstD molecule, HsaAB complex molecule, HsaC molecule or HsaD molecule in the presence of the test compound and the reference compound;

wherein the test compound is an modulator of the KshAB complex molecule, KstD molecule, HsaAB complex molecule, HsaC molecule or HsaD molecule if the activity of the KshAB complex molecule, KstD molecule, HsaAB complex molecule, HsaC molecule or HsaD molecule is greater or less in the presence of the test compound compared to the presence of the reference compound.

This summary of the invention does not necessarily describe all features of the invention. Other aspects and features of the present invention will become apparent to those of ordinary skill in the art upon review of the following description of specific embodiments of the invention.

### BRIEF DESCRIPTION OF THE DRAWINGS

FIG. 1 illustrates a schematic diagram of a catabolic pathway for cholesterol in M. tuberculosis H37Rv. Cholesterol (upper left) rings are labeled A to D and key carbon atoms are numbered. Dashed arrows indicate multiple enzymatic steps. Compound between brackets undergoes non-enzymatic rearrangement. Side chain degradation at C-17 precedes steroid ring degradation. Extracellular cholesterol oxidase (ChoD) transforms cholesterol to 4-cholestene-3-one prior to uptake. Enzymes: KstD, 3-ketosteroid-Δ1-dehydrogenase; KshAB, ketosteroid-9α-hydroxylase (oxygenase and reductase); HsaAB, 3-HSA hydroxylase (oxygenase and reductase); HsaC, 3,4-DHSA dioxygenase; and HsaD. 4,9-DSHA hydrolase. Metabolites: 4-AD, 4-androstene-3,17-dione; 4-ADD, 1,4-androstadiene-3,17-dione; 9-OHADD, 9α-hydroxy-1,4-androstadiene-3,17-dione; 3-HSA, 3-hydroxy-9,10-seconandrost-1,3,5(10)-triene-9,17-dione; 3,4-DHSA, 3,4-dihydroxy-9,10seconandrost-1,3,5(10)-triene-9,17-dione; 4,9-DHSA, 4,5-9,10-diseco-3-ydroxy-5,9,17-trioxoandrosta-1(10),2-diene-4-oic acid; 2-HHD, 2-hydroxyhexadienoate; DOHNAA, 9,17-dioxo-1,2,3,4,10,19-hexanorandrostan-5-oic acid.

FIGS. 2A-C illustrate a schematic diagram of (A) Synthesis of 2,3-dihydroxy-6-methyl-7,8-dihydro-10-Cl stilbene (2,3-DHDS, 6—a reporter substrate for HsaC) and an inhibitor (8). (B) ¹H NMR spectrum of 2,3-DHDS. (C) ¹³C NMR spectrum of 2,3-DHDS.

FIG. 3 illustrates a schematic diagram of synthesis of 2-Cl-3,4-DHSA.

FIG. 4 illustrates a schematic diagram of synthesis preparation of 5-nitro-10-Cl-2,3-DHDS, in accordance with a further embodiment of the present invention.

FIG. 5 illustrates a schematic diagram of synthesis reparation of 2-F-4-AD.

FIG. 6 illustrates a schematic diagram of preparation of 2-Cl-4-AD, in accordance with a further embodiment of the present invention

FIG. 7 illustrates a schematic diagram of preparation of 7,7-dihydroxymethyl-10-Cl-2,3-DHDS, in accordance with a further embodiment of the invention.

FIG. 8 illustrates enzymatic transformation of 6-Me-10-Cl-DHDS (6) to 5-Me-10-Cl-HOPODA (25).

### DETAILED DESCRIPTION

### Mycobacterial Infections

Mycobacterial infections include infections caused by mycobacteria such as M. tuberculosis complex (M. tuberculosis, M. bovis, M. africanum, and M. microti), which cause tuberculosis; M. leprae which causes Hansen's disease or leprosy; M. avium complex (MAC) (M. avium subspecies avium (MAA), subspecies hominis (MAH), and subspecies paratuberculosis (MAP)). MAC, and other nontuberculous mycobacteria (NTM) may cause opportunistic infections in immunocompromised subjects (such as those with HIV) including pulmonary disease resembling tuberculosis, lymphadenitis, skin disease, or disseminated disease.

Diagnosis of mycobacterial infections, including tuberculosis, may be performed using standard techniques such as blood culture, sputum culture, lymph node culture or biopsy, bone marrow culture, stool culture, chest x-ray or CT scan, skin biopsy, etc.

### Genes Involved in Cholesterol Degradation

A suite of genes in M. tuberculosis that are involved in cholesterol degradation have been identified. Table 1 lists several of these genes, together with homologous genes in Rhodococcus RHA1 and M. bovis BCG.

TABLE 1
Annotation of ^{Rhodococcus} RHA1, M. tuberculosis H37Rv
and M. bovis BCG genes assigned to cholesterol pathway.

| | | | | | Annotation of gene | | |
|---|---|---|---|---|---|---|---|
| Gene^{a} | RHA1^{b} | H37Rv^{c} | BCG^{d} | Identity^{e} | product | Best hit^{f} | Identity^{g} |
| mce4F | Ro04703 | Rv3494c | Bcg3558c | 37 | MCE family protein | NA | NA |
| mce4E | Ro04702 | Rv3495c | Bcg3559c | 35 | MCE family protein | NA | NA |
| mce4D | Ro04701 | Rv3496c | Bcg3560c | 43 | MCE family protein | NA | NA |
| mce4C | Ro04700 | Rv3497c | Bcg3561c | 41 | MCE family protein | NA | NA |
| mce4B | Ro04699 | Rv3498c | Bcg3562c | 46 | MCE family protein | NA | NA |
| mce4A | Ro04698 | Rv3499c | Bcg3563c | 41 | MCE family protein | CAA50257 | 32 |
| supB | Ro04697 | Rv3500c | Bcg3564c | 66 | Sterol uptake | AAT51760 | 49 |
| | | | | | permease subunit | | |
| | | | | | (ABC transporter) | | |
| supA | Ro04696 | Rv3501c | Bcg3565c | 72 | Sterol uptake | AAT51759 | 55 |
| | | | | | permease subunit | | |
| | | | | | (ABC transporter) | | |
| choD | Ro04305 | Rv3409c | Bcg3479c | 60 | Cholesterol oxidase | P12676 | 19 |
| hsd4A | Ro04695 | Rv3502c | Bcg3566c | 59 | 17beta- | BAD66689 | 36 |
| | | | | | hydroxysteroid | | |
| | | | | | dehydrogenase | | |
| hsd4B | Ro04531 | Rv3538 | Bcg3602 | 62 | 2-Enoyl acyl-CoA | CAA55037 | 30 |
| | | | | | hydratase | | |
| kshA* | Ro04538 | Rv3526 | Bcg3590 | 59 | Ketosteroid-9alpha- | AAL96829 | 57 |
| | | | | | hydroxylase, | | |
| | | | | | oxygenase | | |
| kshB* | Ro05833 | Rv3571 | Bcg3636 | 54 | Ketosteroid-9alpha- | AAL96830 | 69 |
| | | | | | hydroxylase, | | |
| | | | | | reductase | | |
| kstD | Ro04532 | Rv3537 | Bcg3601 | 62 | 3-Ketosteroid-alpha | AAL82579 | 40 |
| | | | | | 1-dehydrogenase | | |
| hsaB* | Ro04542 | Rv3567c | Bcg3632c | 70 | 3-HSA hydroxylase, | BAC67692 | 16 |
| | | | | | reductase | | |
| hsaC* | Ro04541 | Rv3568c | Bcg3633c | 81 | 3,4-DHSA | BAB15809 | 42 |
| | | | | | dioxygenase | | |
| hsaD* | Ro04540 | Rv3569c | Bcg3634c | 75 | 4,9-DSHA hydrolase | BAC67693 | 31 |
| hsaA* | Ro04539 | Rv3570c | Bcg3635c | 76 | 3-HSA hydroxylase, | BAC67691 | 37 |
| | | | | | oxygenase | | |
| hsaF | Ro04535 | Rv3534c | Bcg3598c | 79 | 4-Hydroxy-2- | P51017 | 48 |
| | | | | | oxovalerate | | |
| | | | | | aldolase | | |
| hsaG | Ro04534 | Rv3535c | Bcg3599c | 85 | Acetaldehyde | BAB97164 | 61 |
| | | | | | dehydrogenase | | |
| hsaE | Ro04533 | Rv3536c | Bcg3600c | 71 | 2-Hydroxypenta | BAB97166 | 59 |
| | | | | | dienoate hydratase | | |
| fadE26 | Ro04693 | Rv3504 | Bcg3568 | 77 | Acyl-CoA | P71539 | 25 |
| | | | | | dehydrogenase | | |
| fadE27 | Ro04692 | Rv3505 | Bcg3569 | 54 | Acyl-CoA | P16219 | 24 |
| | | | | | dehydrogenase | | |
| fadD17 | Ro04691 | Rv3506 | Bcg3570 | 56 | Fatty acid-CoA | Q4LDG0 | 20 |
| | | | | | synthetase | | |
| fadD19 | Ro04689 | Rv3515c | Bcg3578c | 64 | Fatty acid-CoA | AAB87139 | 38 |
| | | | | | ligase | | |
| echA19 | Ro04688 | Rv3516 | Bcg3579 | 73 | Fatty acid-CoA | P31551 | 33 |
| | | | | | hydratase | | |
| ltp4 | Ro04684 | Rv3522 | Bcg3586 | 72 | 3-Ketoacyl-CoA | NA | NA |
| | | | | | thiolase | | |
| ltp3 | Ro04683 | Rv3523 | Bcg3587 | 79 | SCPx related 3- | AAA40098 | 20 |
| | | | | | ketoacyl-CoA | | |
| | | | | | thiolase | | |
| | Ro06698 | NA | NA | NA | Cyclohexanone | AAG01290 | 59 |
| | | | | | monooxygenase | | |
| | Ro06693 | NA | NA | NA | 5-Valerolactone | BAC22650 | 68 |
| | | | | | hydrolase | | |

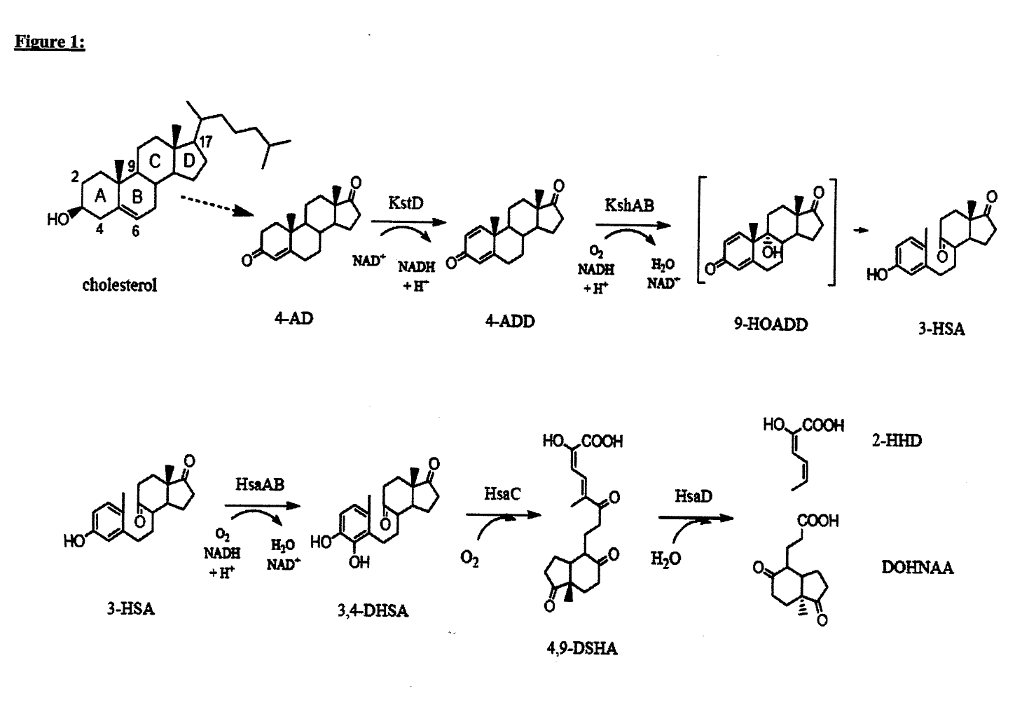

^{a}Name assigned.
^{b}Identification number for an RHA1 gene.
^{c}Identification number for the reciprocal best hit in M. tuberculosis H37Rv.
^{d}Identification number for the reciprocal best hit M. bovis BCG.
^{e}Percent amino acid sequence identity of the RHA1 and H37Rv and BCG orthologues based on full sequence alignment. Nucleotide sequence identity between H37Rv and BCG genes is >98%.
^{f}Accession number of functionally characterized best hit in National Center for Biotechnology Information database.
^{g}Percent amino acid sequence identity of the RHA1 enzyme and its experimentally characterized best hit based on full sequence alignment. NA, not available (either no homologous gene in H37Rv or BCG, or no functionally characterized homolog reported).
*M. tuberculosis gene products indicated in FIG. 1.

Within the cluster of M. tuberculosis genes, several were identified as having roles in various aspects of lipid catabolism, as illustrated in FIG. 1, which is a schematic diagram of a partial cholesterol degradation pathway for M. tuberculosis. In FIG. 1, the successive actions of 3-ketosteroid-Δ1-dehydrogenase (KstD) and 3-ketosteroid-9α-hydroxylase (KshAB) cleave ring B with concomitant aromaticization of ring A, yielding 3-hydroxy-9,10-seconandrost-1,3,5(10)-triene-9,17-dione (3-HSA). 3-HSA is transformed by a hydroxylase, HsaAB, to a catechol, 3,4-dihydroxy-9,10-secoandrost-1,3,5(10)-triene-9,17-dione (3,4-DHSA). HsaC catalyzes the oxygenolytic ring cleavage of 3,4-DHSA, yielding 4,5-9,10-diseco-3-hydroxy-5,9,17-trioxoandrosta-1(10),2-diene-4-oic acid (4,9-DHSA). This is hydrolyzed at a C—C bond to 2-hydroxy-hexa-2,4-dienoate (2-HHD) and 9,17-dioxo-1,2,3,4,10,19-hexanor-androstan-5-oate (DOHNAA). 2-HHD is transformed to central metabolites. M. tuberculosis possesses β-oxidation enzymes to catabolize the side chain of cholesterol. This pathway catabolizes the cholesterol molecule to various metabolites, indicating that the sterol is an important source of energy during survival in the macrophage. Thus, M. tuberculosis genes function in catabolism of steroid rings A and B (transformation of 4-AD to 3—HSA-kstD, kshA, kshB; hydroxylation of 3-HSA (hsaA and hsaB), extradiol cleavage of 3,4 DHSA (hsaC), hydrolysis of the ring-cleaved product, 4,9DHSA (hsaD), β-oxidative degradation of the sidechain (hsd4A, hsd4B, fadD19, fadE26, ltp3, echA19, fad D17, fadE27, ltp4) and further transformation of the DOHNAA metabolite originating from steroid rings C and D—fadE28). Other genes involved in a multi-component cholesterol uptake system were identified (supA, supB, mce4A, mce4B, mce4C, mce4D, mce4E, mce4F).

The gene products of kshA, kshB, hsaA, hsaB, hsaC and hsaD had not been identified in M. tuberculosis before. Moreover, neither the physiological role of these genes nor their participation in the same pathway had been previously recognized.

### KshAB Complex

KshAB complex (ketosteroid-9α-hydroxylase complex) of M. tuberculosis catalyzes the hydroxylation of the polycyclic 3-ketosteroid structure at the 9α position (FIG. 1). The KshAB complex comprises oxygenase KshA (Rv3526; GenBank accession #BX842583—SEQ ID NO:16); GenPept accession #CAB05051—SEQ ID NO: 17) and reductase KshB (GenBank accession #BX842583—SEQ ID NO: 18; GenPept accession #CAB07145—SEQ ID NO: 19). KshAB is a type 1A Rieske nonheme iron oxygenase (RO). ROs catalyze a variety of stereo- and regio-specific reactions and comprise a “Rieske-type” FeS cluster and a mononuclear iron that orchestrates catalysis. Further information on ROs may be found in, for example, FERRARO et al 2005. Biochem. Biophys. Res. Commun. 338:175-190. In alternative embodiments, KshA or KshB nucleic acid or amino acid molecules may include molecules substantially identical to the sequences set forth in SEQ ID NOs: 16 to 19. In alternative embodiments, KshA or KshB nucleic acid or amino acid molecules may include molecules may include homologous molecules from other species or strains. Generally, substrate hydroxylation requires two reducing equivalents, originating from NADPH and transferred to KshA by KshB. Following substrate hydroxylation by KshA, the iron centre and FeS cluster of KshA are oxidized. Reduction of the mononuclear iron centre may be required for product release.

### HsaAB Complex

HsaAB complex (3-hydroxy-9,10-seconandrost-1,3,5(10)-triene-9,17-dione hydroxylase complex) of M. tuberculosis catalyzes the hydroxylation of 3-HSA. The HsaAB complex comprises oxygenase HsaA (Rv3570; GenBank Accession #BX842583—SEQ ID NO: 20; GenPept Accession #CAB07144—SEQ ID NO: 21) and reductase HsaB (Rv3567c; GenBank Accession #BX842583—SEQ ID NO: 22; GenPept Accession #CAB07141—SEQ ID NO: 23). In alternative embodiments, HsaA or HsaB nucleic acid or amino acid molecules may include molecules substantially identical to the sequences set forth in SEQ ID NOs: 20 to 23. In alternative embodiments, HsaA or HsaB nucleic acid or amino acid molecules may include molecules may include homologous molecules from other species or strains.

### HsaC

HsaC (3,4-dihydroxy-9,10-seconandrost-1,3,5(10)-triene-9,17-dione dioxygenase; 3,4-DHSA dioxygenase) (bphC, Rv3568c; EC 1.13.11.39; GenBank Accession #BX842583—SEQ ID NO: 24; GenPept Accession #CAB07142—SEQ ID NO: 25) of M. tuberculosis catalyzes the oxygenolytic cleavage of a catecholic intermediate (FIG. 1). Catechols are cleaved intradiol (between the adjacent —OH groups on the ring) or extradiol (adjacent to the —OH groups). Extradiol enzymes may be dependent on Fe(II). HsaC is a Type I extradiol dioxygenase. Further information on dioxygenases is known in the art, and may be described in, for example Vaillancourt et al. Ring-cleavage dioxygenases. In The Pseudomonads Vol III. Biosynthesis of Macromolecules and Molecular metabolism (Ed. J L Ramos), pp. 359-95, 2004, Kluwer Academic/Plenum Publishers, New York. In alternative embodiments, HsaC nucleic acid or amino acid molecules may include molecules substantially identical to the sequences set forth in SEQ ID NOs: 24 to 25. In alternative embodiments, HsaC nucleic acid or amino acid molecules may include molecules may include homologous molecules from other species or strains.

Generally, the catecholic substrate binds to the Fe(II) in a bidentate manner, displacing solvent ligands and enabling selective deprotonation of one of the two —OH. Binding of the catecholic substrate may activate the Fe(II) for O₂ binding. The mechanism further involves an iron-mediated transfer of an electron from the catechol to the O₂, yielding a semiquinone-Fe(II)-superoxide intermediate, which reacts to give an iron-alkylperoxo intermediate, and under subsequent undergoes alkenyl migration, Criegee rearrangement and O—O bond cleavage to give an unsaturated lactone intermediate and an Fe(II)-bound hydroxide, as described in the art, for example Vaillancourt et al., supra.

### HsaD

HsaD (4,5-9,10-diseco-3-hydroxy-5,9,17-trixoxandrosta-1(10),2-diene-4-oic acid hydrolase; 4,9-DSHA hydrolase) (bphD, Rv3569c; GenBank Accession #BX842583—SEQ ID NO: 26; GenPept Accession #CAB07143—SEQ ID NO: 27) of M. tuberculosis catalyzes the hydrolysis of 4,9-DHSA at a C—C bond (FIG. 1). HsaD belongs to a family of enzymes that hydrolyse vinylogous diketones produced by extradiol cleavage of catechols, such as those produced by HsaC. Further information on hydrolases is known in the art and may be found in, for example, BUGG, 2004. Bioorg Chem 32:367-75. In alternative embodiments, HsaD nucleic acid or amino acid molecules may include molecules substantially identical to the sequences set forth in SEQ ID NOs: 26 to 27. In alternative embodiments, HsaD nucleic acid or amino acid molecules may include molecules may include homologous molecules from other species or strains.

Generally, the enol form of the substrate may undergo an enzyme-catalyzed tautomerization to the keto form before being subject to nucleophilic attack. A serine residue in the active site may act as a base and activate a solvent species to nucleophilic attack. A catalytic histidine residue facilitates both tautomerization and cleavage of the C—C bond.

A “substantially identical” sequence is an amino acid or nucleotide sequence that differs from a reference sequence only by one or more conservative substitutions, as discussed herein, or by one or more non-conservative substitutions, deletions, or insertions located at positions of the sequence that do not destroy the biological function of the amino acid or nucleic acid molecule. Such a sequence can be any integer from 10% to 99%, or more generally at least 10%, 20%, 30%, 40%, 50, 54%, 55%, 59%, or 60%, or at least 65%, 70%, 75%, 76%, 80%, 81%, 85%, 90%, or 95%, or as much as 96%, 97%, 98%, or 99% identical when optimally aligned at the amino acid or nucleotide level to the sequence used for comparison using, for example, the Align Program (Myers and Miller, CABIOS, 1989, 4:11-17) or FASTA. For polypeptides, the length of comparison sequences may be at least 2, 5, 10, or 15 amino acids, or at least 20, 25, or 30 amino acids. In alternate embodiments, the length of comparison sequences may be at least 35, 40, or 50 amino acids, or over 60, 80, or 100 amino acids. For nucleic acid molecules, the length of comparison sequences may be at least 5, 10, 15, 20, or 25 nucleotides, or at least 30, 40, or 50 nucleotides. In alternate embodiments, the length of comparison sequences may be at least 60, 70, 80, or 90 nucleotides, or over 100, 200, or 500 nucleotides. Sequence identity can be readily measured using publicly available sequence analysis software (e.g., Sequence Analysis Software Package of the Genetics Computer Group, University of Wisconsin Biotechnology Center, 1710 University Avenue, Madison, Wis. 53705, or BLAST software available from the National Library of Medicine, or as described herein). Examples of useful software include the programs Pile-up and PrettyBox. Such software matches similar sequences by assigning degrees of homology to various substitutions, deletions, substitutions, and other modifications.

Alternatively, or additionally two nucleic acid sequences may be “substantially identical” if they hybridize under high stringency conditions. In some embodiments, high stringency conditions are, for example, conditions that allow hybridization comparable with the hybridization that occurs using a DNA probe of at least 500 nucleotides in length, in a buffer containing 0.5 M NaHPO₄, pH 7.2, 7% SDS, 1 mM EDTA, and 1% BSA (fraction V), at a temperature of 65° C., or a buffer containing 48% formamide, 4.8×SSC, 0.2 M Tris-Cl, pH 7.6, 1×Denhardt's solution, 10% dextran sulfate, and 0.1% SDS, at a temperature of 42° C. (These are typical conditions for high stringency northern or Southern hybridizations.) Hybridizations may be carried out over a period of about 20 to 30 minutes, or about 2 to 6 hours, or about 10 to 15 hours, or over 24 hours or more. High stringency hybridization is also relied upon for the success of numerous techniques routinely performed by molecular biologists, such as high stringency PCR, DNA sequencing, single strand conformational polymorphism analysis, and in situ hybridization. In contrast to northern and Southern hybridizations, these techniques are usually performed with relatively short probes (e.g., usually about 16 nucleotides or longer for PCR or sequencing and about 40 nucleotides or longer for in situ hybridization). The high stringency conditions used in these techniques are well known to those skilled in the art of molecular biology, and examples of them can be found, for example, in Ausubel et al., Current Protocols in Molecular Biology, John Wiley & Sons, New York, N.Y., 1998, which is hereby incorporated by reference.

As used herein, the term “conservative substitutions” refers to the substitution of one amino acid for another at a given location in the peptide, where the substitution can be made without substantial loss of the relevant function. In making such changes, substitutions of like amino acid residues can be made on the basis of relative similarity of side-chain substituents, for example, their size, charge, hydrophobicity, hydrophilicity, and the like, and such substitutions may be assayed for their effect on the function of the peptide by routine testing.

Conservative substitutions include, without limitation, the following exemplary substitutions:

| Original Amino Acid | Substitution |
|---|---|
| Ala (A) | val; leu; ile |
| Arg (R) | lys; gln; asn |
| Asn (N) | gln; his; asp, lys; gln |
| Asp (D) | glu; asn |
| Cys (C) | ser; ala |
| Gln (Q) | asn; glu |
| Glu (E) | asp; gln |
| Gly (G) | ala |
| His (H) | asn; gln; lys; arg |
| Ile (I) | leu; val; met; ala; phe; |
| | norleucine |
| Leu (L) | norleucine; ile; val; met; |
| | ala; phe |
| Lys (K) | arg; gln; asn |
| Met (M) | leu; phe; ile |
| Phe (F) | leu; val; ile; ala; tyr |
| Pro (P) | ala |
| Ser (S) | thr |
| Thr (T) | ser |
| Trp (W) | tyr; phe |
| Tyr (Y) | trp; phe; thr; ser |
| Val (V) | ile; leu; met; phe; ala; |
| | norleucine |

### Inhibitors and Methods of Synthesis Thereof

Genes involved in steroid metabolism are useful targets for therapeutic agents or drugs. In some embodiments, such genes are essential to M. tuberculosis survival in the host, and/or have no human homologue. In alternative embodiments, where the genes encode oxygenases, the reaction of the oxygenases with their substrate analogues can result in production of reactive oxygen species (ROS). Inhibitors of enzymes involved in steroid metabolism, e.g., cholesterol degradation by mycobacteria, inhibit steps of an essential metabolic pathway. Accordingly, such inhibitors are useful for the treatment of mycobacterial infections, such as tuberculosis.

As used herein, an ‘inhibitor’ refers to an agent that interferes with the normal activity of an enzyme, e.g., KshA, KshB, HsaA, HsaB, HsaC or HsaD. Enzyme inhibition may be reversible or irreversible. The extent of reversible inhibition may be dependent on the concentration of the inhibitor—high inhibitory activity occurs when the inhibitor concentration is high, and vice versa. Irreversible inhibition renders the enzyme inactive, for example, through a covalent modification of a functional group on the enzyme. Reversible inhibition may be classified as competitive, non-competitive, uncompetitive or mixed. Competitive inhibitors compete for the active site of the enzyme and often resemble the substrate. Non-competitive inhibition occurs when the inhibitor interacts with another site of the enzyme, distal to the substrate binding site. This interaction may result in a change of shape, or prevent a change of shape for example. Uncompetitive inhibition occurs from the combination of the enzyme-substrate complex with the inhibitor. Mixed inhibition occurs when multiple types of inhibition occur at the same time—the effect may or may not be cooperative. X-ray crystallographic analysis may be used to provide detailed insight into how a specific inhibitor, substrate or substrate analog binds, and this information used to develop better inhibitors or substrate analogues. Details on the kinetics of various enzyme reactions and how to determine them are known in the art, and may be found in, for example, Methods in Enzymology, various volumes (Academic Press), or in Creighton T E. Proteins; Structural and Molecular Properties 1993. WH Freeman and Company, New York. An inhibitor may be a ‘substrate analogue’ i.e., an agent that is similar to a naturally occurring substrate for an enzyme. A substrate analogue may also be an alternative substrate for an enzyme and not necessarily inhibit the enzyme. In some embodiments, a substrate analogue may be used as a control. The product of the enzymatic transformation of the substrate analogue may be a metabolite, e.g., a toxic metabolite, or may be a synthetic substance. Use of an enzyme to convert a substrate analogue to a desired product is referred to as ‘biotransformation’. The enzyme may function in isolation, or may be expressed by an organism, such as a bacterium. The enzyme may be a normal component of the bacterium, or may be a genetically modified construct expressed in a heterologous organism.

An inhibitor includes, without limitation, any compound that is capable of reducing the activity of an enzyme by at least 10%, 20%, 30%, 40%, 50%, 60%, 70%, 80%, 90%, or 100%, using techniques as described herein or known in the art. Inhibitors may include any one or more of the compounds described herein.

As used herein ‘reactive oxygen species’ (ROS) refer to chemical moieties comprising at least one oxygen atom having an unpaired electron. Examples of ROS include H₂O₂ (hydrogen peroxide), OCl⁻ (hypochlorite) OH. (hydroxyl radical), and O₂⁻ (superoxide anion). Other examples of ROS are known to those of skill in the art. ROS may be produced as a by-product of cellular respiration or metabolism, for example the respiratory chain in the mitochondrion, or may be synthesized in phagocytic cells, for example, neutrophils by NADPH oxidase or myeloperoxidase. ROS are strong oxidants and as exemplified, may be used by cells to kill invading microorganisms. If not controlled in an isolated vesicle, or by cellular enzymes that degrade ROS (for example superoxide dismutase, which converts two superoxide anions into hydrogen peroxide and oxygen), the ROS may result in biological toxicity to the cell or organism. For example, the biological toxicity of superoxide is due to its capacity to inactivate iron-sulfur cluster containing enzymes (which are critical in a wide variety of metabolic pathways), thereby liberating free iron in the cell, which can undergo Fenton-chemistry and generate the highly reactive hydroxyl radical. In its HO₂ form, superoxide can also initiate lipid peroxidation of polyunsaturated fatty acids. As such, superoxide is a main cause of oxidative stress.

The mechanisms by which activated oxygen species are generated during the enzymatic transformation of preferred substrates are known in the art. For example, the reaction may be controlled such that the activated oxygen intermediate reacts essentially exclusively with the substrate to produce the intended transformation product. In a tightly coupled reaction, for example, the organic substrate, O₂ and NADH may be consumed in 1:1:1 stoichiometry. Some substrate analogues may cause uncoupling of the enzymatic reaction.

Substrates that cause uncoupling of the reaction in the enzymes involved in steroid catabolism may generate toxic ROS. Additionally, such substrates may cause inhibition of an essential metabolic pathway in M. tuberculosis, or may cause a net metabolic energy drain on the bacterium by futile consumption of NADH in the uncoupled reaction.

A Compound Reference Table (Table 2) showing the structures of exemplary compounds referred to herein, using the corresponding compound name, is provided below. Other exemplary compounds include stereoisomers, diastereomers, enantiomers and tautomers of the illustrated compounds of Table 2.

TABLE 2
Compound Reference Table

| Compound name | Structure |
|---|---|
| 2-chloro-4-androstene-3,17- dione (2-Cl-4-AD) (α and β enantiomers) | 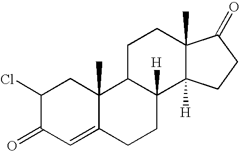 |
| 1-nitro-4-androstene-3,17- dione (1-nitro-4-AD) (α and β enantiomers) | 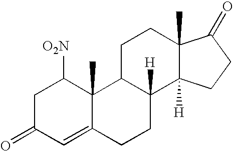 |
| 4-fluoro-4-androstene-3,17- dione (4-F-4-AD) (α and β enantiomers) | 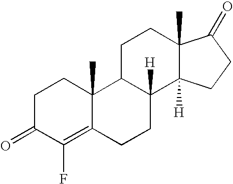 |
| 2-chloro-3-hydroxy-9,10- seconandrost-1,3,5(10)- triene-9,17-dione (2-Cl-3-HSA) | 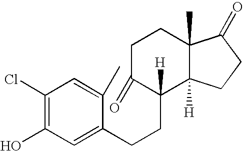 |
| 1-nitro-10-chloro-3- hydroxy-6-methyl-7,8- dihydrostilbene (1-nitro-10-Cl-3-HDS) | 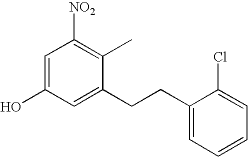 |
| 4-fluoro-3-hydroxy-9,10- seconandrost-1,3,5(10)- triene-9,17-dione (4-F-3-DHSA) | 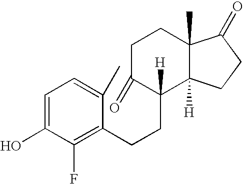 |
| 7-hydroxymethyl-7- hydroxyethyl-10-Cl-3- hydroxy-6-methyl-7,8- dihydrostilbene (7-hydroxymethyl-7- hydroxyethyl-10-Cl-3-HDS) | 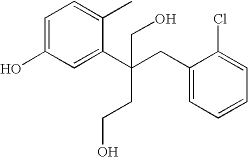 |
| 2-chloro-3,4-dihydroxy-9,10- seconandrost-1,3,5(10)- triene-9,17-dione (2-Cl-3,4-DHSA) | 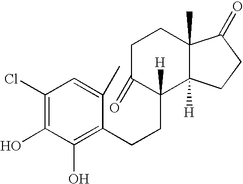 |
| 1-nitro-2,3-dihydroxy-6- methyl-7,8-dihydrostilbene (1-nitro-2,3-DHDS) | 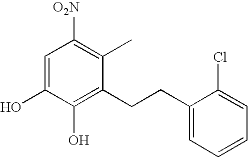 |
| 7,7-dihydroxymethyl 3,4- dihydroxy-9,10-seconandrost- 1,3,5(10)-triene-9,17-dione (7,7-dihydroxymethyl 3,4-DHSA) | 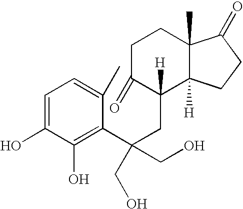 |
| 7-hydroxymethyl-7- hydroxyethyl-10-Cl-2,3- dihydroxy-6-methyl-7,8- dihydrostilbene (7-hydroxymethyl-7- hydroxyethyl- 10-Cl-2,3-DHDS) | 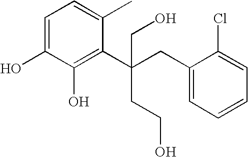 |
| 7,7-dihydroxymethyl-10- Cl-2,3-dihydroxy-6-methyl- 7,8-dihydrostilbene (7,7-dihydroxymethyl-10-Cl- 2,3-DHDS) | 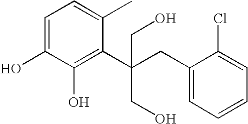 |
| 3-chlorocatechol (3-cc) | 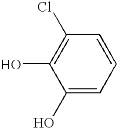 |
| 2′,6′-diCl-2,3- dihydroxybiphenyl (2′,6′-diCl DHB) | 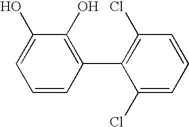 |
| 3-chloro-4,5-9,10-diseco-3- hydroxy-5,9,17- trioxoandrosta-1(10),2-diene- 4-oic acid (3-Cl-4,9-DSHA) | 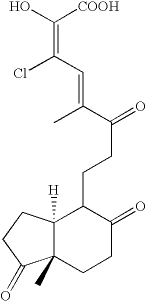 |
| (2E,4Z)-8-(2-chlorophenyl)- 2-hydroxy-4,5-dimethyl-6- oxoocta-2,4-dienoic acid (4,5-diMe-10-Cl-HOPODA) | 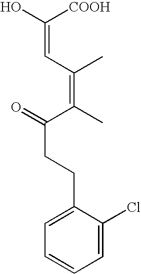 |

### KshAB

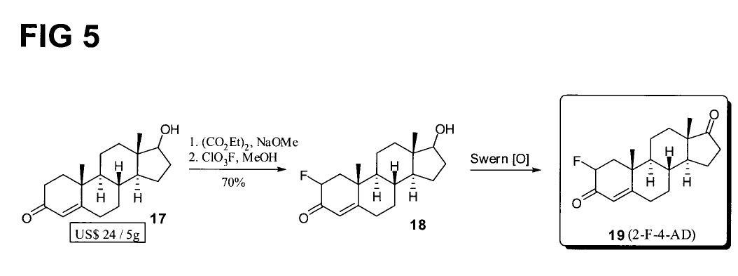

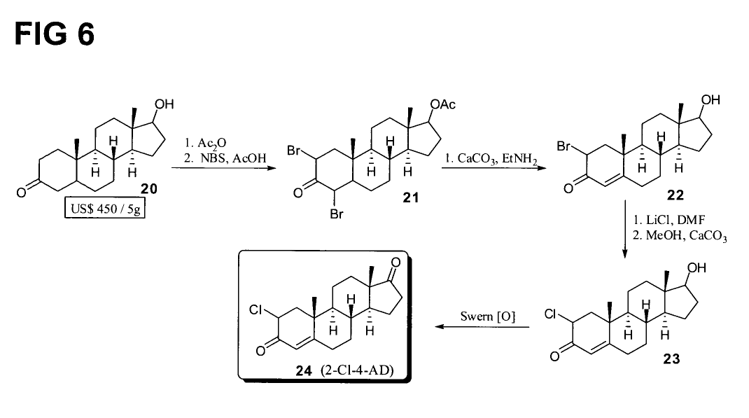

In an alternative embodiment of the invention, inhibitors for KshA or KshB include analogues of 4-ADD that bind to the enzyme and/or cause uncoupling of the enzymatic reaction, for example compounds having similarity to 4-ADD such as 2-Cl-4-AD (FIG. 6), 2-F-4-AD (FIG. 5) and 1-nitro-4-AD, or stereoisomers, diastereomers, enantiomers and tautomers thereof, including racemic mixtures.

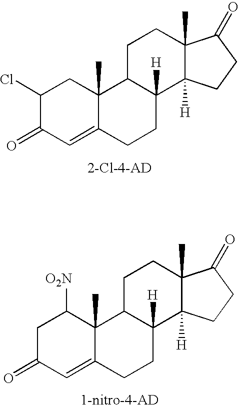

In an alternative embodiment of the invention, inhibitors of KshAB compete with the steroid-like substrate for the enzyme's active site, and also lead to uncoupling of O₂ and NADH utilization from hydroxylation of the compound.

### HsaAB

In an alternative embodiment of the invention, inhibitors of HsaAB include substituted phenols according to Formula I. Electron-withdrawing substituents may occur on the phenolic ring, and/or a substituent may physically occlude the binding of O₂ to HsaAB or prevent the reaction of the activated oxygen intermediate with the bound phenol at position R7 of Formula 1 (see, for example, the enzymatic step carried out by HsaAB in FIG. 1). Inhibitors of HsaAB may compete with the phenolic substrate for the enzyme's active site, or may lead to uncoupling of O₂ and NADH utilization.

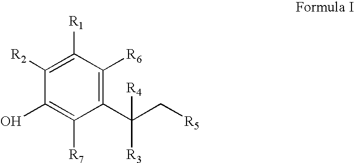

R₁, R₂, R₆ may independently be an electron-withdrawing substituent group, including Cl, F, Br, I, —NO₂, —CH═O, —CR═O, —COOH, COOR, CONR₂, COCl, —CX₃; where X is a halogen (Cl, Br, F, I), —CN, —SO₃H, —NH₃⁺, NR₃⁺). R₇ may be any small substituent, from one to 10 atoms. R₃ and R₄ may independently be H, —CH₂OH, —CH₂CH₂OH, —CH₂Cl, —CH₂CH₂Cl, —CH₂NH₂, or —CH₂CCl₃. etc. R₅ may be any ring, including cycloalkanone, phenyl, purine, pyrimidine, bicycloalkanone, and the like.

As used herein, a ‘bicycloalkane’ refers to an alkane comprising two aliphatic rings that share two carbon atoms. The shared carbon atoms may be adjacent, or may be separated by a ‘bridge’ carbon. The aliphatic rings may be of 3, 4, 5, 6, 7 or more members. Ring members may be all carbon, or may include oxygen, nitrogen or other heteroatoms. A ‘bicycloalkanone’ is a bicycloalkane having at least one C═O substituent on at least one carbon of the bicycloalkane. The aliphatic rings may be in the cis- or the trans-orientation.

For example, in a substrate analogue of 3-HDS (3-hydroxy-6-methyl-7,8-dihydrostilbene), R₁, R₂, R₃, R₄, R₇═H; R₅=bicycloalkanone; R₆═—CH₃.

In another example, an inhibitor of HsaAB may be 2-Cl-3-HSA or 1-nitro-10-Cl-3-HDS, or stereoisomers, diastereomers, enantiomers or tautomers thereof, including racemic mixtures.

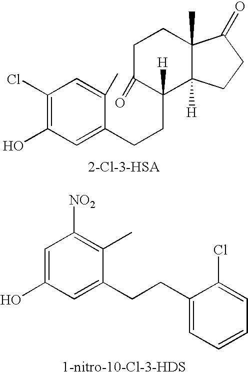

In another example, the inhibitors may be 4-F-3-DHSA, 7-hydroxymethyl-7-hydroxyethyl-3-HDS, or 7-hydroxymethyl-7-hydroxyethyl-10-Cl-3-HDS, and stereoisomers, diastereomers, enantiomers and tautomers thereof, including racemic mixtures.

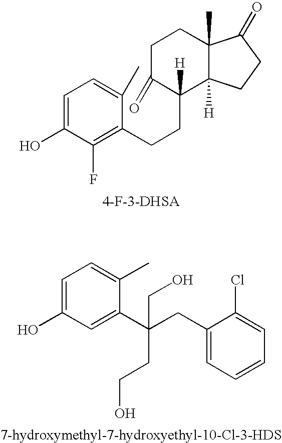

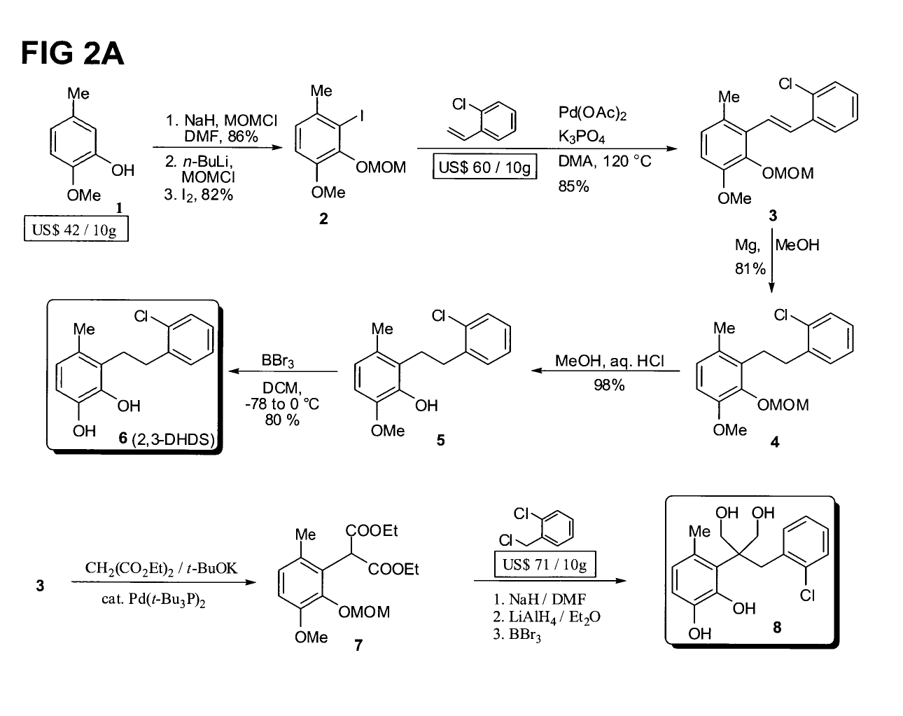

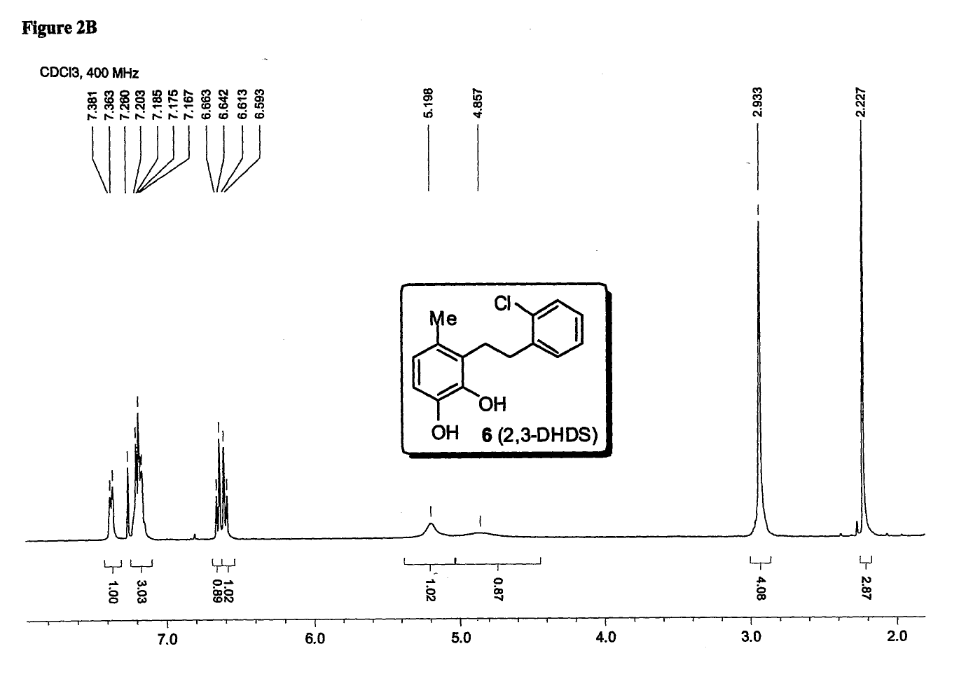

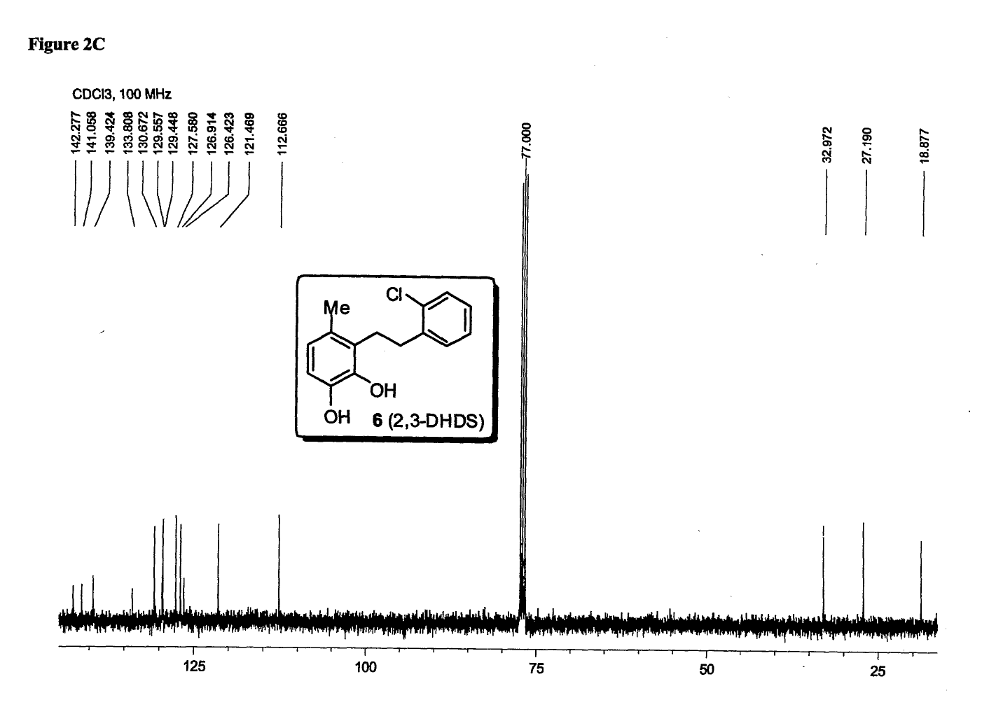

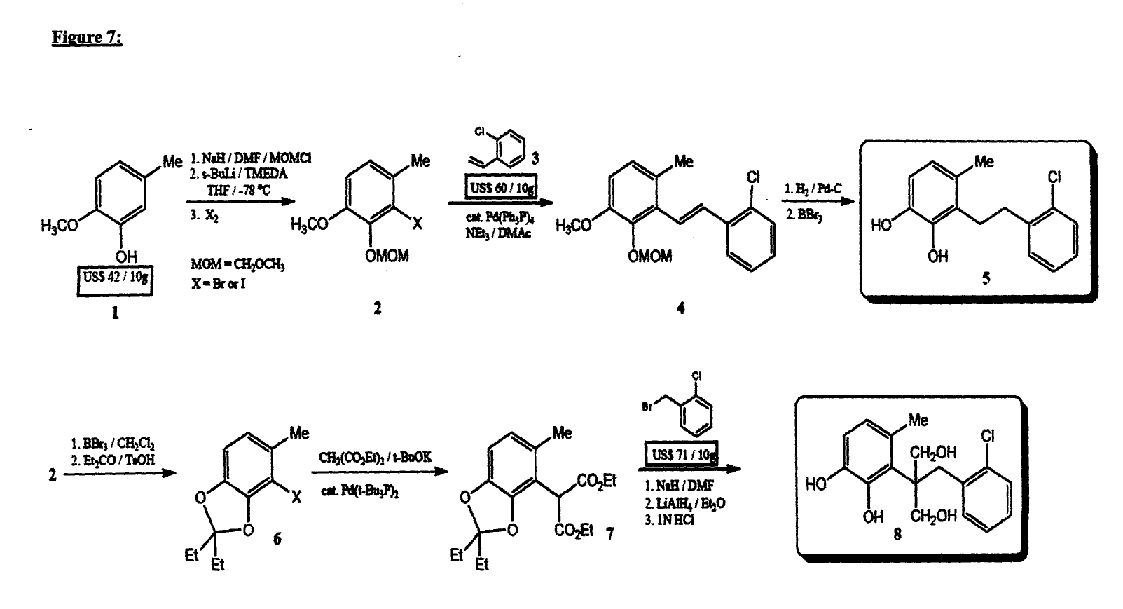

For the synthesis of alternative substrates, 2-chlorostyrene may be used in the preparation of various compounds using arylbromide Heck coupling partners, as used for preparation of DHSA analogues (see, for example, FIG. 7). In an alternative embodiment of the invention, metabolic precursors of the inhibitors may generate the inhibitory phenolic ring. For example, the corresponding substituted cholesterol or 4-AD can be transformed to the requisite inhibitory 3-HSA in vivo by M. tuberculosis enzymes. For example, the starting material may be 3-bromo-4-methylphenol, and employ a method similar to that illustrated in FIG. 2a, for example, further comprising a Hartwig coupling with buturolactone, anionic benzylation and a reductive ring opening to diol.

### HsaC

In an alternative embodiment of the invention, inhibitors of HsaC include substituted catechols according to Formula II. In an alternative embodiment of the invention, inhibitors of HsaC compete with the catecholic substrate for the enzyme's active site or inactivate the enzyme. A non-limiting example of this inactivation may be through increasing the rate of oxidation of the active site Fe(II) to Fe(III), which may result in the production of superoxide, O₂⁻. Alternatively, HsaC may be inhibited by a substrate analogue that has a group or groups (electron-withdrawing or otherwise) that sterically restricts binding of O₂ to the active site Fe(II) of HsaC, or may sterically interfere with the productive reaction between the activated oxygen intermediate and C₄.

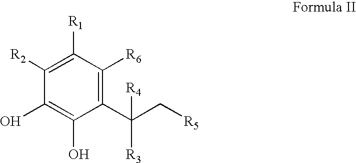

R₁, R₂, R₆ may independently be an electron-withdrawing substituent group, including Cl, F, Br, I, —NO₂, —CH═O, —CR═O, —COOH, COOR, CONR₂, COCl, —CX₃ where X is a halogen (Cl, Br, F, I), —CN, —SO₃H, —NH₃⁺, NR₃⁺). R₃ and R₄ may independently be H, —CH₂OH, —CH₂CH₂OH, —CH₂Cl, —CH₂CH₂Cl, —CH₂NH₂, or —CH₂CCl₃. etc. R₅ may be any ring, including cycloalkanone, phenyl, purine, pyrimidine, bicycloalkanone, heterocycle, and the like.

For example, in a substrate analogue 3,4-DHSA, R₁, R₂, R₃, R₄═H; R₅=bicycloalkanone; R₆═—CH₃. In another example, the substrate analogue is 2,3-DHDS (2,3-dihydroxy-6-methyl-7,8-dihydrostilbene), where R₁, R₂, R₃, R₄═H; R₅=2-Cl phenyl; R₆═—CH₃.

In another example an inhibitor of HsaC may be 3-chlorocatechol, 2-Cl-3,4-DHSA or 1-nitro-2,3-DHDS, or stereoisomers, diastereomers, enantiomers or tautomers thereof, including racemic mixtures.

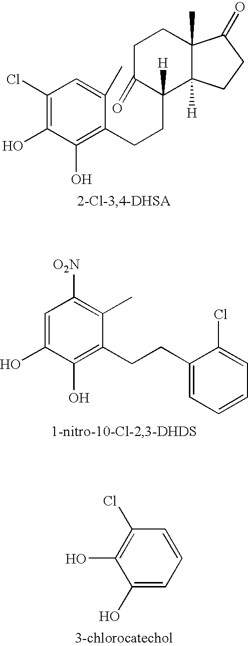

For example, such substituents may occur at R₃ or R₄ of Formula II, and may include —CH₂OH, —CH₂CH₂OH, —CH₂Cl, —CH₂CH₂Cl, —CH₂NH₂ or —CH₂CCl₃.

In another example, the inhibitors may be 7,7-dihydroxymethylated 3,4-DHSA, 2′,6′-diCl DHB, or 7-hydroxy-methyl-7-hydroxyethyl-2,3-DHDS, and stereoisomers, diastereomers, enantiomers and tautomers thereof, including racemic mixtures.

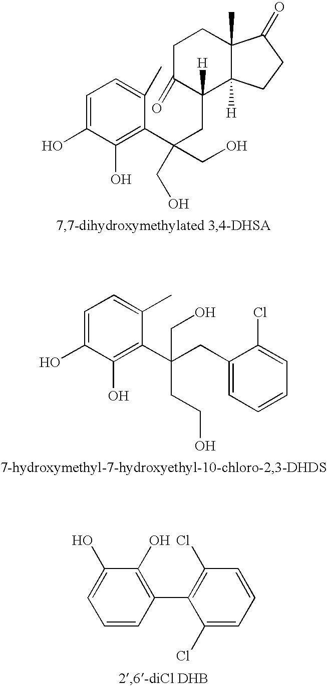

Substrate analogues for HsaC, for example 3,4-DHSA and a dihydrostilbene derivative, 2,3-DHDS (FIG. 2a) may be synthesized, for example, using metalation-cross coupling methodologies, as described in the Examples.

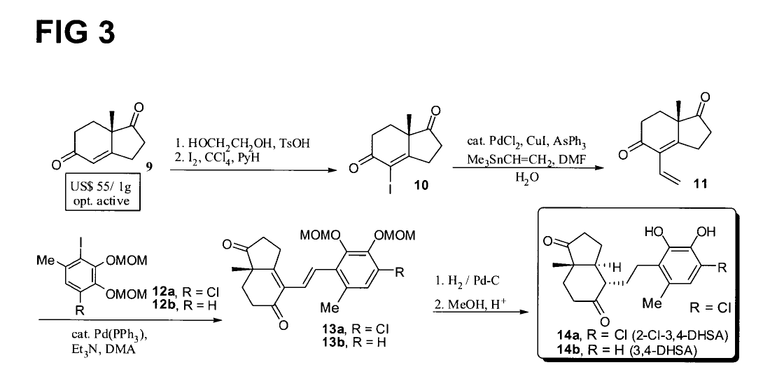

The methods outlined in FIGS. 2A and 3 are examples for application to the preparation of other compounds, for example 3,4-DHSA or other fluorinated and chlorinated derivates such as 1-F-3,4-DHSA, 1-Cl-3,4-DHSA, or 2-F-3,4-DHSA. The choice of such compounds depends on kinetic, biochemical and structural data. Synthesis of DHSAs may be initiated from the diketone (structure 9) or catechol or phenol derivatives, e.g. for 3,4-DHSA from 3-Br-4-Me-diMOM catechol, prepared by, for example, directed ortho metallation (DoM).

In an alternative embodiment of the invention, metabolic precursors of the inhibitors may be used to generate the inhibitory catechol in vivo. For example, the corresponding substituted cholesterol, 4-AD, 3-HSA or 3-HDS can be transformed to the requisite inhibitory catechol in vivo by M. tuberculosis enzymes.

### HsaD

In an alternative embodiment of the invention, inhibitors of HsaD include substituted 2-hydroxy-6-oxo-2,4-dienoates (HODAs) according to Formula III. For example in a substrate analogue 4,9-DSHA, R₁₃, R₁₄═H; R₁₅═—CH₃ and R₁₇=bicycloalkanone. In another example, 5-Me-10-Cl-HOPODA, R₁₃, R₁₄═H; R₁₅═—CH₃ and R₁₇=2-Cl phenyl. Alternatively, R₁₇ may be any ring, for example, a cycloalkanone, phenyl, purine or pyrimidine attached at any available site on these systems.

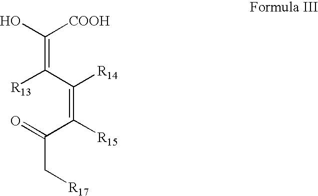

One or more of R₁₃, R₁₄ and R₁₅ may independently be, for example, Cl, F, Br, I, —NO₂, —CH═O, —CR═O, —COOH, COOR, CONR₂, COCl, —CX₃, H, —CH₂OH, —CH₂CH₂OH, —CH₂Cl, —CH₂CH₃, —CH₂CH₂Cl, —CH₂NH₂, or —CH₂CCl₃; where X is a halogen (Cl, Br, F, I), —CN, —SO₃H, —NH₃⁺, NR₃⁺. R₁₇ may be a halogen (Cl, F, Br, I), or any ring, including cycloalkanone, phenyl, purine, pyrimidine, bicycloalkanone, heterocycle, or the like, which may further comprise a halogen substituent group.

In another embodiment of the invention, one or more of R₁₃, R₁₄ and R₁₅ may be substituents that sterically affect the binding of the HODA in the active site of HsaD, or alter or induce tautomerization. Examples of such inhibitors may include 3-Cl-4,9-DSHA and 4,5-diMe-10-Cl-HOPODA, or stereoisomers, diastereomers, enantiomers or tautomers thereof, including racemic mixtures.

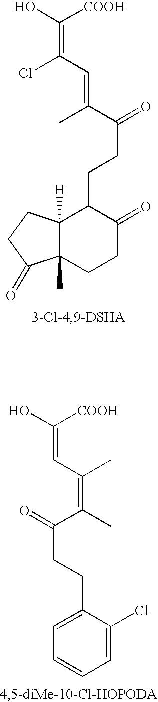

In another embodiment of the invention, metabolic precursors may be used to generate the inhibitory HODA in vivo. For example, the corresponding substituted cholesterol-4-AD, 3-HSA, 3,4-DHSA, 3-HDS or 2,3-DHDS—can be transformed to the requisite inhibitory HODA in vivo by M. tuberculosis enzymes.

### Biotransformation Methods for Substrate Analogues and Inhibitors

In an alternative embodiment of the invention, substrate analogues and inhibitors may be generated from commercially available or synthesized compounds using biotransformation. Such biotransformations may be performed using whole cells or purified enzymes, using methods known to those of skill in the art, for example VOISHVILLO et al 2004. Appl Biochem. Microbiol. 40:463-469; FERNANDES et al 2003 Enzyme Microb. Technol 32: 688-705; BOYD et al 2006. Org. Biomol. Chem. 4:181-192) with modifications as necessary, also known to those of skill in the art.

For example, whole cells may be used for biotransformations involving multiple components, such as HsaAB and KshAB. The strains may be E. coli or Pseudomonas expressing specific mycobacterial enzymes, for example, or may be disruption mutants of RHA1 generated by disruption of one or more genes as known in the art (van der Geize & Dijkhuizen (2004)Curr Opin Microbiol 7, 255-61). Alternatively, multiple specific enzymes may be expressed in a single type of bacteria using described strategies, for example, that of TAN 2001. Protein Expression and Purification 21:224-234. Cells may be grown to mid-log phase and gene expression induced. Cells may be harvested at maximal expression, and resuspended in an appropriate buffer, for example, a buffer comprising 0.1% glucose and the substrate to be biotransformed. Biotransformation may be monitored by HPLC, or spectrophotometrically for ring-cleaved compounds. The necessary methods will be known to those of skill in the art, and may be modified as necessary, also known to those of skill in the art.

In another embodiment of the invention, 4,9-DHSA and related analogues may be produced from the corresponding 3,4-DHSAs using purified HsaC for biotransformation. The necessary methods will be known to those of skill in the art, and may be modified as necessary, also known to those of skill in the art.

In another embodiment of the invention, the substrate analogues or inhibitors may be generated by M. tuberculosis by biotransformation in situ. For example 2-F-4-AD or 2-F-3-HSA may be administered to a patient, test subject or assayed as described. The enzymes of M. tuberculosis in the steroid catabolic pathway may biotransform these substrate analogues into an inhibitor of a downstream enzyme, for example, 2-F-3,4-DHSA, which inhibits HsaC.

The compounds described herein, or their pharmaceutically acceptable salts may contain one or more asymmetric centers and may thus give rise to enantiomers, diastereomers, and other stereoisomeric forms that may be defined, in terms of absolute stereochemistry, as (R)- or (S)- or, as (D)- or (L)- for amino acids. The present invention is meant to include all such possible isomers, as well as, their racemic and optically pure forms. Optically active (+) and (−), (R)- and (S)-, or (D)- and (L)-isomers may be prepared using chiral synthons or chiral reagents, or resolved using conventional techniques, such as reverse phase HPLC. When the compounds described herein contain olefinic double bonds or other centers of geometric asymmetry, and unless specified otherwise, it is intended that the compounds include both E and Z geometric isomers. Likewise, all tautomeric forms are also intended to be included.

Suitable conditions for carrying out one or more synthetic steps required to synthesize chemical compounds and inhibitors are described herein or may be determined by reference to publications directed to methods used in synthetic organic chemistry. Texts and treatises that detail the synthesis of reactants useful in the preparation of compounds of the present invention, will also provide suitable conditions for carrying out a synthetic step according to the present invention. Suitable texts and treatises that describe the synthesis of reactants useful in the preparation of compounds, or provide references to articles that describe the preparation, include without limitation, “Synthetic Organic Chemistry”, John Wiley & Sons, Inc., New York; S. R. Sandier et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Specific and analogous reactants may also be identified through the indices of known chemicals prepared by the Chemical Abstract Service of the American Chemical Society, which are publicly available. Chemicals that are known but not commercially available in catalogs may be prepared by custom chemical synthesis houses, where many of the standard chemical supply houses (e.g., those listed above) provide custom synthesis services.

### Methods for Identifying Inhibitors

In general, test compounds are identified from large libraries of both natural products or synthetic (or semi-synthetic) extracts or chemical libraries according to methods known in the art. Those skilled in the field of drug discovery and development will understand that the precise source of test extracts or compounds is not critical. Accordingly, virtually any number of chemical extracts or compounds can be screened using the exemplary methods described herein or known in the art. Examples of such extracts or compounds include, but are not limited to, plant-, fungal-, prokaryotic- or animal-based extracts, fermentation broths, and synthetic compounds, as well as modification of existing compounds. Numerous methods are also available for generating random or directed synthesis (e.g., semi-synthesis or total synthesis) of any number of chemical compounds, including, but not limited to, small molecule, saccharide-, lipid-, peptide-, and nucleic acid-based compounds. Synthetic compound libraries are commercially available. Alternatively, libraries of natural compounds in the form of bacterial, fungal, plant, and animal extracts are commercially available from a number of sources, including Biotics (Sussex, UK), Xenova (Slough, UK), Harbor Branch Oceanographic Institute (Ft. Pierce, Fla., USA), and PharmaMar, MA, USA. In addition, natural and synthetically produced libraries are produced, if desired, according to methods known in the art, e.g., by standard extraction and fractionation methods. Furthermore, if desired, any library or compound is readily modified using standard chemical, physical, or biochemical methods.

When a crude extract is found to inhibit an enzyme, e.g., KshA, KshB, HsaA, HsaB, HsaC or HsaD, further fractionation of the positive lead extract is necessary to isolate chemical constituents responsible for the observed effect. Thus, the goal of the extraction, fractionation, and purification process is the careful characterization and identification of a chemical entity within the crude extract having inhibitory activities. The same assays for the detection of activities in mixtures of compounds can be used to purify the active component and to test derivatives thereof. Methods of fractionation and purification of such heterogeneous extracts are known in the art. If desired, compounds shown to be useful agents for treatment of mycobacterial diseases, e.g., tuberculosis are chemically modified according to methods known in the art. Alternatively, test compounds may be prepared on rational drug design concepts, based on known substrates or substrate analogues of an enzyme, e.g., KshA, KshB, HsaA, HsaB, HsaC or HsaD, using techniques described herein or known in the art. In an alternative embodiment, inhibitors for KshA or KshB can be screened using standard methods using, for example, 4-androstene-3,17-dione (4-AD) or 1,4-androstadiene-3,17-dione (4-ADD) as a positive control.

Compounds identified as being of therapeutic, prophylactic, diagnostic, or other value may be subsequently analyzed using an animal model as described herein or known in the art.

### In Vitro Testing

Various methods, as described herein or known in the art, may be used to test or measure the ability of a test compound to inhibit an enzyme e.g., KshA, KshB, HsaA, HsaB, HsaC or HsaD, produce a desired synthetic product, or produce a toxic metabolite, such as an ROS. The gene encoding a desired enzyme may be cloned and expressed using methods known in the art. The enzyme may be isolated and purified to varying degrees using various chromatographic, and related methods known in the art. Some enzymes may require special conditions to maintain activity, for example anaerobic conditions, or specific temperatures, pH or organic additives. Methods of determining such special conditions and modifying known methods will be within the ability of one of skill in the art. Production of ROS may be assayed using kits and methods known in the art. For example, peroxides may be assayed using the Amplex Red Reagent (Invitrogen). Alternatively, oxidative activity in live cells may be assayed using, for example the Image-IT LIVE Green Reactive Oxygen Species Detection Kit (Invitrogen). Other examples of methods, kits, assays and techniques will be known to those of skill in the art.

An enzyme's activity may be measured using various methods known in the art including spectrophotometry, polarography and HPLC. Specific assay conditions such as pH, temperature, and methods of detection, for example, may relate to the enzyme of interest. Determination of the necessary assay conditions will be within the skill of one knowledgeable in the art.

A “test compound” is any naturally-occurring or artificially-derived chemical compound. Test compounds may include, without limitation, peptides, polypeptides, synthesised organic molecules, naturally occurring organic molecules, and nucleic acid molecules. A test compound can “compete” with a known compound such as a natural substrate of KshA, KshB, HsaA, HsaB, HsaC or HsaD by, for example, interfering with cholesterol degradation (as for example illustrated in FIG. 1); or by interfering with any biological response induced by the known compound. In alternative embodiments, a test compound inhibits activity of KshA, KshB, HsaA, HsaB, HsaC or HsaD when compared to a natural substrate of any one or more of these enzymes.

Test compounds may be tested for their ability to inhibit growth of mycobacteria. Mycobacterial species and strains suitable for growth assays may include Mycobacterium bovis Bacille Calmette-Guerin (BCG) and M. tuberculosis H37Rv. Bacterial species or strains closely related to these mycobacteria may also be used, for example Rhodococcus RHA1, or bacterial species engineered to produce mycobacterial enzymes (e.g., E. coli). For example, a compound to be tested may be incorporated into the culture medium and the growth rate of the bacterium compared to a control. Alternatively, a compound to be tested may be incorporated into a porous matrix, such as a filter disk and placed on a ‘lawn’ of bacteria. As the test compound diffuses out of the disk into the surrounding medium, a zone of inhibition may occur. Comparison of the size of the zone of inhibition may indicate the relative efficacy of the test compound as an inhibitor at the provided concentration.

As an example, a 50% growth inhibitory concentration (GIC₅₀) assay may be employed to determine the concentration of a test compound that inhibits M. tuberculosis growth by 50% in vitro (Gruppo et al. 2006. Antimicrobial Agents and Chemotherapy 50:1245-1250).

As M. tuberculosis infects macrophages in the mammalian host, toxicity of the inhibitors to the host cells may also be assayed. Macrophages infected with mycobacterial species may be cultured in vitro using established methods, and assessed for toxic effects (cytotoxicity) when exposed to the inhibitors. Methods, kits or assays for assessing cytotoxicity may include, for example, the LIVE/DEAD Viability/Cytotoxicity Kit (Invitrogen), or the MTT assay (various suppliers). Examples of published methods and assays that may be useful to assess cytotoxicity include those of PICK et al 2004 Biological Procedures 6:220-225 and PICK et al 2006. Med. Microbiol. 55:407-415.

### In Vivo Testing

In vivo bioavailability, efficacy, or activity of a test compound may be assessed in a subject.

As used herein, a ‘subject’ refers to a human or other animal. A subject may have an active or latent mycobacterial infection, for example an M. tuberculosis infection, or may be uninfected. A subject may be a human, a non-human primate such as a chimpanzee, or a monkey, or may be a mammal such as a mouse, rat, guinea pig, rabbit, cow, sheep, dog, cat, horse. A subject may be a clinical patient, a clinical trial volunteer, an experimental animal, etc. A subject may be immunologically naïve (i.e. not previously exposed to a mycobacterial strain or combination of strains), or may have been vaccinated or otherwise exposed to a mycobacterial strain or combination of strains, for example a previous inoculation or mycobacterial infection. The subject may be suspected of having or at risk for having a mycobacterial infection, e.g., tuberculosis; be diagnosed with a mycobacterial infection, e.g., tuberculosis; or be a control subject that is confirmed to not have a mycobacterial infections, e.g., tuberculosis. Diagnostic methods for mycobacterial infections, e.g., tuberculosis, and the clinical delineation of mycobacterial infection, e.g., tuberculosis, diagnoses are known to those of ordinary skill in the art.

Test compounds may be administered to a subject by any of several methods, including oral gavage, injection, topical application, inhalation, etc, and the blood and tissue distribution assessed. For example, the subject is administered a dose of the agent, and blood samples taken at various times following dosing, and the drug concentration in the serum measured by conventional means. Gruppo et al 2006 Antimicrobial Agents and Chemotherapy 50:1245-1250 describe an exemplary method for determining in vivo bioavailability of orally administered agents in a mouse model.

Various animal models for tuberculosis are described by Gupta et al., 2005. (Tuberculosis 85:277-293). Selection of a particular animal model may be dependent on the aspect of the infection to be studied, immunological response, vaccine efficacy, therapeutic agent efficacy, method of infection, and the like. A mouse model for rapid in vivo screening of agents using an aerosol infection route is described by Lenaerts et al 2003 (Antimicrobial Agents and Chemotherapy 47:783-785). A low-dose aerosol infection model is described by Kelly et al 1996. (antimicrobial Agents and Chemotherapy 40:2809-2812. An intravenous infection model using outbred mice is described by, for example Cynamon et al 1999. (Antimicrobial Agents and Chemotherapy 43:1189-1191). Guinea pigs have also demonstrated suitability for testing anti-tuberculosis agents (Gupta et al 2005 (Tuberculosis 85:277-293), being particularly similar to humans with respect to some aspects of M. tuberculosis infection. While the genome of M. tuberculosis has been sequenced, many of the gene products are not known. Gene disruption studies M. tuberculosis may provide a way to investigate the role of a particular gene in the context of an in vitro or in vivo model. Various gene disruption methods in slow growing mycobacteria such as M. tuberculosis and M. bovis BCG are reported, including short or long linear DNA fragments as allelic exchange substrates (Azad et al., 1996. Proc. Natl. Acad Sci USA 93:4787-4792; Kalpana et al., 1991. Proc. Natl. Acad Sci USA 88:5433-5437; Reyrat et al., 1995 Proc. Natl. Acad Sci USA 92:8786-8772; Balasubramanian et al., 1996. J. Bacteriol 178:273-279); suicide vectors (Berthet et al., 1998 Science 282:759-762; Fitzmaurice & Kolattukudy, 1998 J. Biol Chem 273:8033-8039; Knipfer et al., 1997 Plasmid 37:129-140; Parish et al., 1999 Microbiology 145-3497-3503; Pavelka & Jacobs, 1996. J. Bacteriol 178:6496-6507; Pelicic et al., 1996a. Mol Microboiol 20:919-925; Pelicic et al. 1996b FEMS Microbiol Lett 144:161-166; Pelicic et al, 1997. Proc. Natl. Acad Sci USA 94:10955-10960; Sander et al., 1995. Mol Microbiol 16:991-1000) and a two-step selection method using selectable and counterselectable markers (Knipfer et al., 1997, supra; Pelicic et al., 1996a, supra; Hinds et al., 1999 Microbiology 145:519-527; Parish et al., 1999, supra; Parish & Stoker, 2000. Microbiology 146:1969-1975; Pavelka & Jacobs, 1999, J. Bacteriol 181:4780-4789). A specialized transduction method for generating marked and unmarked targeted gene disruptions has also been described (Bardarov et al 2002. Microbiology 148:3007-3017).

The gene-disrupted mycobacterial strains may be tested in mice to assess infection attenuation. For example, immunocompromised mice may be infected intravenously with an infection dose of the mycobacterial strain and observed for infection progress and survival time. An infection dose may vary depending on the test subject, but may generally be estimated as the number of colony forming units (CFU) of unmutated mycobacteria necessary to establish an infection. For example, intravenous infection of SCID mice with 10⁵ CFU M. tuberculosis H37Rv produces an infection that kills the mice in about 21-28 days.

Other general methods, protocols and details for cloning and expression of enzymes, enzyme assays, immunologic studies and the like may also be found in, for example, Sambrook, J., Maniatis, T., Fritsch, E. F. in Molecular Cloning: A Laboratory Manual, Second Edition, Cold Spring Harbor Laboratory Press, Cold Spring Harbor, N.Y. 1989; Ausubel, et al., Current Protocols in Molecular Biology, John Wiley & Sons, New York, N.Y.; Coligan et al., eds., Current Protocols in Protein Science, John Wiley & Sons, New York, N.Y.; Harlow and Lane, Antibodies: A Laboratory Manual, Cold Spring Harbour, N.Y.; Coligan, et al., eds., Current Protocols in Immunology, John Wiley & Sons, New York, N.Y.; Methods in Enzymology, various volumes (Academic Press); Creighton T E. Proteins; Structural and Molecular Properties 1993. WH Freeman and Company, New York.

Agents according to various embodiments of the invention may be tested for GIC₅₀, bioavailability and/or therapeutic efficacy according the methods exemplified above.

### Methods for Treating Mycobacterial Infections

Compounds of the invention can be provided alone or in combination with other compounds (for example, nucleic acid molecules, small molecules, peptides, or peptide analogues), in the presence of a liposome, an adjuvant, or any pharmaceutically acceptable carrier, in a form suitable for administration to mammals, for example, humans, cattle, sheep, etc. If desired, treatment with a compound according to the invention may be combined with more traditional and existing therapies for mycobacterial infections, e.g., tuberculosis. For example, treatment with one or more of an inhibitor of KshA, KshB, HsaA, HsaB, HsaC or HsaD may be combined with one or more of isoniazid (INH), rifampin (RIF), pyrazinamide or ethambutol. Compounds according to the invention may be provided chronically or intermittently. “Chronic” administration refers to administration of the agent(s) in a continuous mode as opposed to an acute mode, so as to maintain the initial therapeutic effect (activity) for an extended period of time. “Intermittent” administration is treatment that is not consecutively done without interruption, but rather is cyclic in nature.

Compositions comprising an inhibitor according to any of the various embodiments of the invention, may be administered as a dose from about 0.1 ug/kg to about 20 mg/kg (based on the mass of the subject), or any amount therebetween, for example from about 1 ug to about 2000 ug/ml or any amount therebetween, about 10 ug to about 1000 ug or any amount therebetween, or about 30 ug to about 1000 ug or any amount therebetween. For example, a dose of about 0.1, 0.5, 1.0, 2.0, 5.0, 10.0 15.0, 20.0, 25.0, 30.0, 35.0, 40.0, 50.0 60.0, 70.0, 80.0, 90.0, 100, 120, 140, 160 180, 200, 250, 500, 750, 1000, 1500, 2000, 5000, 10000, 20000 ug, or any amount therebetween may be used. In alternative embodiments, a suitable dosage range may be any integer from 0.1 nM-0.1 M, 0.1 nM-0.05M, 0.05 nM-15 μM or 0.01 nM-10 μM

It is to be noted that dosage values may vary with the severity of the condition to be alleviated. For any particular subject, specific dosage regimens may be adjusted over time according to the individual need and the professional judgment of the person administering or supervising the administration of the compositions. Dosage ranges set forth herein are exemplary only and do not limit the dosage ranges that may be selected by medical practitioners. The amount of active compound(s) in the composition may vary according to factors such as the disease state, age, sex, and weight of the individual. Dosage regimens may be adjusted to provide the optimum therapeutic response. For example, a single bolus may be administered, several divided doses may be administered over time or the dose may be proportionally reduced or increased as indicated by the exigencies of the therapeutic situation. It may be advantageous to formulate parenteral compositions in dosage unit form for ease of administration and uniformity of dosage.

In general, compounds of the invention should be used without causing substantial toxicity. Toxicity of the compounds of the invention can be determined using standard techniques, for example, by testing in cell cultures or experimental animals and determining the therapeutic index, i.e., the ratio between the LD₅₀ (the dose lethal to 50% of the population) and the LD100 (the dose lethal to 100% of the population). In some circumstances however, such as in severe disease conditions, it may be necessary to administer substantial excesses of the compositions.

An “effective amount” of an agent as used herein refers to the amount of agent required to have a prophylactic, palliative or therapeutic effect when administered to a subject. For therapeutic or prophylactic compositions, the compounds may be administered to an individual in an amount sufficient to stop or slow a mycobacterial infection, e.g., tuberculosis. A “therapeutically effective amount” refers to an amount effective, at dosages and for periods of time necessary, to achieve the desired therapeutic result, such as elimination or reduction in the severity of a mycobacterial infection, e.g. tuberculosis. A therapeutically effective amount of a compound may vary according to factors such as the disease state, age, sex, and weight of the individual, and the ability of the compound to elicit a desired response in the individual. Dosage regimens may be adjusted to provide the optimum therapeutic response. A therapeutically effective amount is also one in which any toxic or detrimental effects of the compound are outweighed by the therapeutically beneficial effects. A “prophylactically effective amount” refers to an amount effective, at dosages and for periods of time necessary, to achieve the desired prophylactic result, such as prevention of a mycobacterial infection, e.g. tuberculosis. Typically, a prophylactic dose is used in subjects prior to or at an earlier stage of disease, so that a prophylactically effective amount may be less than a therapeutically effective amount.

Compositions comprising an agent according to various embodiments of the invention may be formulated with any of a variety of pharmaceutically acceptable excipients, frequently in an aqueous vehicle such as Water for Injection, Ringer's lactate, isotonic saline or the like. Pharmaceutically acceptable excipients include, for example, salts, buffers, antioxidants, complexing agents, tonicity agents, cryoprotectants, lyoprotectants, suspending agents, emulsifying agents, antimicrobial agents, preservatives, chelating agents, binding agents, surfactants, wetting agents, non-aqueous vehicles such as fixed oils, or polymers for sustained or controlled release. See, for example, Berge et al. (1977. J. Pharm Sci. 66:1-19), or Remington—The Science and Practice of Pharmacy, 21st edition. Gennaro et al editors. Lippincott Williams & Wilkins Philadelphia (both of which are herein incorporated by reference). In some embodiments, the excipients may be carboxymethylcellulose or poly-L-lysine. Molecular weight, concentrations and methods of preparation of such excipients may be found in, for example, U.S. Pat. No. 4,349,538.

Compositions comprising an agent according to various embodiments of the invention may be administered by any of several routes, including, for example, subcutaneous injection, intraperitoneal injection, intramuscular injection, intravenous injection, epidermal or transdermal administration, mucosal membrane administration, orally, nasally, rectally, or vaginally. See, for example, Remington—The Science and Practice of Pharmacy, 21st edition. Gennaro et al editors. Lippincott Williams & Wilkins Philadelphia. Carrier formulations may be selected or modified according to the route of administration.

Compositions comprising an agent according to various embodiments of the invention may be provided in a unit dosage form, or in a bulk form suitable for formulation or dilution at the point of use.

Compositions comprising an agent according to various embodiments of the invention may be administered to a subject in a single-dose, or in several doses administered over time. Dosage schedules may be dependent on, for example, the subject's condition, age, gender, weight, route of administration, formulation, or general health. Dosage schedules may be calculated from measurements of adsorption, distribution, metabolism, excretion and toxicity in a subject, or may be extrapolated from measurements on an experimental animal, such as a rat or mouse, for use in a human subject. Optimization of dosage and treatment regimens are discussed in, for example, Goodman & Gilman's The Pharmacological Basis of Therapeutics 11th edition. 2006. L L Brunton, editor. McGraw-Hill, New York, or Remington—The Science and Practice of Pharmacy, 21 st edition. Gennaro et al editors. Lippincott Williams & Wilkins Philadelphia.

Compositions according to various embodiments of the invention may be provided in a unit dosage form, or in a bulk form suitable for formulation or dilution at the point of use.

Compositions according to various embodiments of the invention may be administered to a subject in a single-dose, or in several doses administered over time. Dosage schedules may be dependent on, for example, the subject's condition, age, gender, weight, route of administration, formulation, or general health. Dosage schedules may be calculated from measurements of adsorption, distribution, metabolism, excretion and toxicity in a subject, or may be extrapolated from measurements on an experimental animal, such as a rat or mouse, for use in a human subject. Optimization of dosage and treatment regimens are discussed in, for example, Goodman & Gilman's The Pharmacological Basis of Therapeutics 11th edition. 2006. L L Brunton, editor. McGraw-Hill, New York, or Remington—The Science and Practice of Pharmacy, 21 st edition. Gennaro et al editors. Lippincott Williams & Wilkins Philadelphia.

### Kits

In accordance with another aspect of the invention, there is provided a kit for treating a mycobacterial infection, such as tuberculosis, in a subject. The kit may include a pharmaceutical composition comprising a compound that inhibits at least one step in a cholesterol catabolic pathway in a mycobacterium, together with instructions for using a pharmaceutical composition for the treatment of a mycobacterial infection, for example tuberculosis. The instructions may include, for example, dose concentrations, dose intervals, preferred administration methods or the like.

In the context of the present invention, the terms “treatment,”, “treating”, “therapeutic use,” or “treatment regimen” as used herein may be used interchangeably are meant to encompass prophylactic, palliative, and therapeutic modalities of administration of the compositions of the present invention, and include any and all uses of an agent according to various embodiments of the invention that remedy a disease state, condition, symptom, sign, or disorder caused by a mycobacterial infection. Any prevention, amelioration, alleviation, reversal, or complete elimination of an undesirable disease state, symptom, condition, sign, or disorder associated with a mycobacterial infection, is encompassed by the present invention. A treatment may comprise administration of an effective amount of a composition as described herein, comprising an agent according to various embodiments of the invention.

### Methods

Bacterial growth. H37Rv was grown at 37° C. for 30 days on 7H11 solid medium plus 2 mM cholesterol, 2 mM AD or no organic substrate (control). H37Rv was also grown on phospholipid vesicles plus or minus cholesterol in PS liquid mineral medium. The vesicles contained diphosphatidyl choline, diphosphatidyl ethanolamine, diphosphatidyl glycerol, and cholesterol in molar ratios of either 46.8:5.5:2.8:45 or 85:10:5:0.

RHA1 was grown at 30° C. on a shaker in one of two minimal media: W minimal salt medium (Seto et al 1995. Appl Environ. Microbiol. 61:3353-3358) plus 20 mM pyruvate or 2 mM cholesterol or a similar medium supplemented with a different mineral solution (Patrauchan et al 2005. J. Bacterial 187:4050-4063) plus cholesterol, AD, pyruvate, or benzoate as indicated. RHA1 cells were harvested at midlog phase (OD600 of 1.0 for pyruvate and 2.0 for cholesterol).

Bacillus Calmette—Guerin was grown at 37° C. on a tube roller (10 rpm) in screw-capped 15-ml vials filled with 10 ml of liquid medium containing 0.5 g/ml asparagine, 1 g/ml KH2PO4, 2.5 g/ml Na2PO4, 10 mg/l MgSO4.7H2O, 0.5 mg/liter CaCl2, 0.1 mg/liter ZnSO4, 50 mg/liter ferric ammonium citrate, and 0.5 ml/liter Triton wR1339 (Tyloxapol) (Schnappinger et al 2003. J Exp Med 198:693-704) plus the indicated amount of cholesterol, 10 mM pyruvate, or 10 mM glucose. Total protein content of cultures was determined in cells disrupted by sonication (10 cycles of 30 s at 6 μm) by using the Bradford protein assay (BioRad, Hercules, Calif.) and BSA as standard.

RNA Extraction and Microarray. RNA was isolated from RHA1 as described (Goncalves et al 2006. Appl Environ Microbiol 72:6183-6193). RNA was similarly isolated from BCG except that both the RNeasy Plus and RNeasy Mini Kits (Qiagen, Valencia, Calif.) were used, and the sample was treated with 2 units of TURBO DNase (Ambion, Austin, Tex.). The RHA1 transcriptome was analyzed by using indirectly labeled cDNA and a microarray containing 70-mer probes for 8,313 genes as described (Goncalves, supra). Data were analyzed by using GeneSpring (Agilent Technologies, Santa Clara, Calif.) and MeV 3.1 (The Institute for Genomic Research, Rockville, Md.). For each condition, RNA was extracted from each of three independently grown cultures. Data were averaged and normalized by using Locally Weighted Linear Regression (Lowess). Details of the microarray design, transcriptomic experimental design and transcriptomic data have been deposited in the National Center for Biotechnology Information Gene Expression Omnibus (www.ncbi.nlm.nih.gov/geo).

Quantitative RT-PCR. RT-PCR was performed as described (Goncalves, supra) with TaqMan probes, and cDNA was synthesized by using the Thermo-Script RT-PCR System (Invitrogen, Carlsbad, Calif.) and random hexamers. (Table 4) The gene-encoding DNA polymerase IV and σA were used as internal standards in the multiplex reactions performed by using RHA1 and BCG cDNA, respectively (Goncalves, supra). The Ct values were normalized (ΔCt) by subtracting those of the internal standard. Significant differences in ΔCt values were tested by using a two-sample t test assuming unequal variances. Relative fold differences were calculated as 2^{-ΔΔCt}, where ΔΔCt=ΔCtₜᵣₑₐₜₘₑₙₜ−ΔCt_{control}.

TABLE 4
Probes and primers used in RT-PCR

| | SEQ | | |
|---|---|---|---|
| | ID | | |
| Name | NO: | Use | Sequence |
| hsaCR-for3 | 7 | qRT-PCR | TCCGCAAGAAGGTCAAGATGT |
| | | of | |
| | | hsaCRHA1 | |
| hsaCR-rev3 | 8 | qRT-PCR | TCTTCATGTAGAACGACAGCATCA |
| | | of hsaC | |
| | | RHA1 | |
| hsaCR-TP | 9 | Taqman | 6FAM-CCGTGACGTCAACG-MGB |
| | | probe | |
| | | hsaC RHA1 | |
| kshAB-for | 10 | qRT-PCR | TCCTCATCGAAGGGTCCAACT |
| | | of kshA | |
| | | BCG | |
| kshAB-rev | 11 | qRT-PCR | GGATGTAGAAGAAGTGCGCCATAT |
| | | of kshA | |
| | | BCG | |
| kshAB-TP | 12 | Taqman | 6FAM-CATCATCGACAACGTC-MGB |
| | | probe | |
| | | kshA BCG | |
| sigAB-for | 13 | qRT-PCR | TTCGCGCCTACCTCAAACAG |
| | | of | |
| | | sigABCG | |
| sigAB-rev | 14 | qRT-PCR | TCAGCTGCGTGGCGTACA |
| | | of | |
| | | sigABCG | |
| sigAB-TP | 15 | Taqman | 6FAM-TAGCGCTGCTCAACG-MGB |
| | | probe | |
| | | sigABCG | |

### Enzyme Cloning and Expression

The hsaC gene was amplified by PCR using Expand High Fidelity™ DNA polymerase (Roche Applied Sciences, Laval, P. Q., Canada) and cloned essentially as described for bphK₃₀ (FORTIN, 2006 J. Bacteriol vol 188). Briefly, hsaC was amplified using M. tuberculosis H37Rv genomic DNA and primers Hcmt-F (CGACTAGCATATGAGCATCCGGTCGC—SEQ ID NO: 1) and Hcmt-R (CGGGATCCCTGAGCCGACATCGTTTG—SEQ ID NO: 2). HsaD was amplified using M. tuberculosis H37Rv genomic DNA and primers Hdmt-F (CGACGTACATATGACAGCTACCGAGGAATTG—SEQ ID NO: 3) and Hdmt-R (CAGGATCCTCATCTGCCACCTCCCAG—SEQ ID NO: 4). The amplicons were digested with NdeI and BamHI, cloned into similarly digested pT7-7, and their respective nucleotide sequences confirmed by sequencing to yield pT7HC1. HsaC was produced using E. coli GJ1158 transformed with pT7HC1, as described by FORTIN et al. 2005. Evolutionarily Divergent Extradiol Dioxygenases Possess Higher Specificities for PCB Metabolites. J. Bacteriol. 187, 415-421, for use in in vitro inhibitor assays.

Gene Replacement and Deletion. The hsaC and hsaA genes were independently replaced in RHA1 with an apramycin resistance marker, apraR, using a procedure in which the gene was first replaced in a fosmid by using lambda-RED-based methodology and then in RHA1 by using the modified fosmid and allelic exchange (PATRAUCHAN ET AL 2005. J. Bacteriol 187:41050-4063). The parent fosmid, RF00128O15, contained 38.3 kb of RHA1 DNA including the hsaADCB cluster. Guidelines and methods for designing primers to be used in such experiments are known in the art. The oligonucleotides used to generate the resistance cassette used to replace hsaC were hsaC-for1 (TCTGGCCACCGATTTCCTTCTGGACGGGGGTAAGTGATGATTCCGGGGATCCG TCGACC—SEQ ID NO:5) and hsaC-rev1 (CCTCGGCCGCGACGGCCCCGTCCCCGGTCACCTCACTCATGTAGGCTGGAGC TGCTTC—SEQ ID NO: 6). The oligonucleotides used to generate the resistance cassette used to replace hsaA were designed in a similar manner to those used for hsaC. Gene replacements and deletions were verified by using a series of PCRs using: (i) primers matching sequences within the target gene(s), (ii) primers matching sequences flanking the target gene, and, when appropriate, (iii) primers matching a region within apraR.

Enzyme Assays. HsaCH37Rv and HsaDH37Rv activities in cellular extracts were measured by following the formation (HsaC) or consumption (HsaD) of the ring-cleaved product on a Cary 5000 spectrophotometer (Varian, Walnut Creek, Calif.) equipped with a thermojacketed cuvette holder, essentially as described for biphenyl catabolic enzymes (Vaillancourt et al 1998. J. Biol Chem 273:34887-34895). Experiments were performed by using 20 mM 3-[4-(2-hydroxyethyl)-1-piperazinyl]propanesulfonic acid, 80 mM sodium chloride, pH 8.0 at 25.0_{—}0.1° C. Concentrations of 4,9-DSHA (ε₃₉₂=7.64 mM⁻¹ cm⁻¹) and HOPDA (ε₄₃₄=32.5 mM⁻¹ cm⁻¹) were monitored at 392 and 434 nm, respectively. Initial velocities were determined from a least-squares analysis of the linear portion of the progress curves by using the kinetics module of Cary software. Steady-state rate equations were fit to data as described (Vaillancourt, supra).

Metabolite Preparation and Characterization. Culture supernatant was acidified by using 0.5% orthophosphoric acid then extracted twice with 0.5 volume of ethyl acetate. The ethyl acetate extracts were pooled, dried with anhydrous magnesium sulfate, and evaporated to dryness with a rotary evaporator. The residue was dissolved in a 7:3 mixture of methanol/water containing 0.5% phosphoric acid and purified by HPLC with a 2695 separation module (Waters, Milford, Mass.) and a Prodigy 10-μm ODS-Prep column (21.2×250 mm; Phenomenex, Torrance, Calif.). Metabolites were eluted by using the same methanol/water solvent at a flow rate of 5 ml/min. The eluate was monitored at 280 nm. The retention time of the major metabolite was ˜21 min. The fractions containing this metabolite were pooled, added to 10 volumes of water, and extracted as described above. The metabolite was derivatized by using Sylon BFT (Supelco, Bellefonte, Pa.) and analyzed by using a 6890 gas chromatograph (Agilent Technologies) and 5973N mass-selective detector (Agilent Technologies) in electron ionization mode. The extinction coefficient of 4,9-DHSA was determined with an oxygraph assay (Fortin et al 2005. J. Bacteriolo 187:415-421).

### EXAMPLES

The following examples are illustrative of some of the embodiments of the invention described herein, and are not to be considered limiting to the scope of the invention.

### Example 1

### Mutational Analysis of Cholesterol Catabolic Genes

To substantiate the predicted role of HsaC (gene product of hsaC) in catalyzing the extradiol cleavage of 3,4-dihydroxy-9,10-seconandrost-1,3,5(10)-triene-9,17-dione (3,4-DHSA), a catechol, hsaC was deleted in RHA1. In liquid media, the hsaC-mutant grew on cholesterol at a rate that was 60% that of the WT strain and developed a pink color. By contrast, growth on pyruvate was not affected. The pink color was consistent with the accumulation and nonenzymatic oxidation of a catechol. To identify the catechol, metabolites were extracted from the supernatant of hsaC-cells incubated in the presence of cholesterol. HPLC analysis revealed a major metabolite, which, when derivatized with trimethylsilane (TMS), yielded a compound with a molecular ion m/z=460. The molecular ion and its fragmentation pattern corresponded to those for TMS-derivatized 3,4-DHSA. Transformation of HsaC_{H37Rv} with the metabolite yielded a product with a pH-dependent spectrum essentially identical to that reported for 4,5-9,10-diseco-3-hydroxy-5,9,17-trioxoandrosta-1(10),2-diene-4-oic acid (4,9-DSHA) (ε₃₉₂=7.64 mM⁻¹·cm⁻¹ at pH 8.0), confirming the metabolite's identity as 3,4-DHSA (FIG. 1).

### Example 2

### The Catalytic Activities of HsaCH37Rv and HsaDH37Rv

HsaC_{H37Rv} and HsaD_{H37Rv} were heterologously expressed in E. coli, and their steady-state kinetic parameters were evaluated with cell extracts. As summarized in Table 3, the enzymes preferentially transformed cholesterol metabolites as compared with biphenyl metabolites. Specifically, cell extracts containing HsaC_{H37Rv} catalyzed the extradiol cleavage of 3,4-DHSA with an apparent specificity 44-fold higher than for DHB. Similarly, extracts containing HsaD_{H37Rv} catalyzed the hydrolysis of 4,9-DSHA with an apparent specificity 34-fold higher than for 2-hydroxy-6-oxo-6-phenylpentadienoate (HOPDA) (Table 3). Equivalent extracts prepared from cells that contained the empty vector did not detectably transform either the steroid or biphenyl metabolites.

TABLE 3
Steady-state kinetic parameters of HsaC_{H37Rv} and
HsaD_{H37Rv} for steroid and biphenyl metabolites

| Enzyme | Substrate | Km, μM | Vmax, μM^{s-1} | Vmax/Km, s − 1 |
|---|---|---|---|---|
| HsaC_{H37Rv} | 3,4-DHSA | 0.9 (0.5) | 12 (4) | 790 (370) |
| | DHB | 8.5 (0.8) | 2.5 (0.4) | 18 (3) |
| HsaD_{H37Rv} | 4,9-DSHA | 4 (1) | 0.06 (0.02) | 1.0 (0.2) |
| | HOPDA | 19 (6) | 0.009 (0.003) | 0.028 (0.007) |

Parameters were normalized to the amount of cellular extract (milligrams of protein content) used in the assays. Values in parentheses represent standard errors.

### Example 3

### Cholesterol Catabolism in M. Bovis Bacillus Calmette-Guerin (BCG)

BCG was grown in a liquid minimal medium containing asparagine, citrate, and Triton, the final growth yield of BCG was proportional to the initial concentration of cholesterol in the medium. Thus, in medium supplemented with 0, 0.25, and 0.5 mM cholesterol, respectively, the overall protein yields were 22+/−7, 46+/−9, and 70+/−4 μg/ml. Further modification of the medium to reflect host factors or improve the availability of the cholesterol to the bacterium may improve growth.

To investigate whether the predicted cholesterol catabolic pathway was involved in this growth of BCG, quantitative RT-PCR analyses were performed on kshA and hsaC with sigA as a control. Normalized transcript levels were significantly higher in cultures growing on cholesterol (n=4) than on glucose (n=5) for both kshA (P<0.005) and hsaC (P=0.05), with relative fold differences of 3.7 and 2.4, respectively. Similar results were observed when comparing cholesterol- to pyruvate-grown cells. The relative fold differences for kshA and hsaC were very similar to the expression ratios determined for these genes (4.6 and 2.1, respectively) using the microarray to compare RHA1 growing on cholesterol versus on pyruvate, although, a slightly higher fold difference was determined for hsaC in RHA1 with quantitative RT-PCR.

### Example 4

### M. tuberculosis uses Cholesterol and 4-AD as Growth Substrates

The mycobacterial steroid degradation pathway illustrated in FIG. 1 is rich in oxygenases, including four of deduced function and two associated cytochromes P450 of unknown function. Consistent with the identification of this pathway, H37Rv grew on each of cholesterol and 4-androstene-3,17-dione (4-AD). Colonies grew on solid medium with cholesterol or 4-AD as the sole organic substrate, but did not grow on control medium lacking cholesterol. In liquid culture, H37Rv grew faster in medium supplemented with phospholipid vesicles containing cholesterol than in the same medium supplemented with phospholipid vesicles containing no cholesterol.

### Example 5

### Synthetic Methods for HsaC Inhibitors

To obtain 2,3-DHDS (structure 6), commercially available structure 1 (3-methoxy-5-methylphenol: Aldrich Chemical Co.) (FIG. 2A) was converted into an intermediate MOM derivative allowing a directed ortho metalation (DoM) (Wang et al 2005. Tetrahedron. 61:259; Yao et al 2003. J Org Chem 68:7528-7531) and iodination reaction sequence to form the corresponding aryl iodide 2 (Weeratunga et al Can J Chem 65:2019-2023). Compound 2 was then subjected to Heck coupling conditions, (Wang, supra; Yao supra), to afford the stilbene 3 which was reduced (4) (Schertl et al 2001. Archiv der Pharmazie 334:125; Profitt et al 1979. J. Org Chem 44:3972-3974) and deprotected (5) (Furukawa et al 1986. J Chem Soc Chem Comm 1234-1235) to give the requisite catechol 6. The latter was purified by silica gel chromatography and its identity was confirmed by ¹H and ¹³C NMR (FIG. 2b, c). ˜90 mg of 2,3 DHDS at >99% purity was obtained.

Inhibitor 8 (FIG. 2A), is prepared starting with common intermediate 3. The synthetic step to give 7 uses Hartwig methodology (Fillion et al 2005. J Org Chem 70:1316-1327); other steps are described in Katz et al 2003. J. Am. Chem Soc 125:13948-13949; Hahn et al 1991. S. Chem Ber 124:487-491).

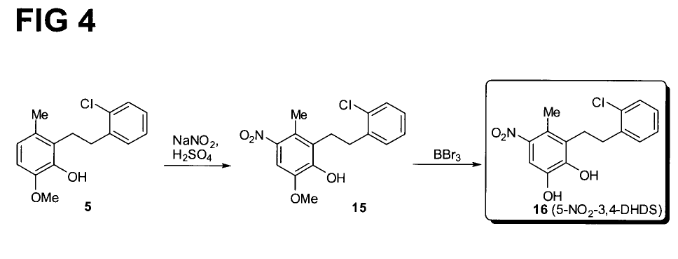

Halogenated and nitrated catechol derivatives of 3,4-DHSA and/or 2,3-DHDS, e.g., 2-Cl-3,4-DHSA (14a, FIG. 3) and 5-nitro-2,3-DHDS (16, FIG. 4)(Samajdar et al 2000. Tetrahedron Lett 41:8017-8020. Synthesis of 14a starts with the commercially available, optically active 9 [Wieland-Miescher ketone, available from Shering Plough AG] and the step to 11 and 13 respectively involve adoption of Stille (Blanchfield et al 2004. Aust J Chem 57:673-676) and Heck (Beletskaya et al 2000. Chem Rev 100: 3009-3066; Kuchezov et al 1964. Izv. Akad. Nauk. SSSR. Ser. Khim 8:1456-1463) cross-coupling protocols; other steps are analogous to known methods (Blanchfield supra; Kuchezov, supra; Macklin and Snieckus 2005. Directed ortho and remote metalation Handbook of C—H Transformations. G Dyker (ed), Wiley-VCH, Weinhem pp 106-118). Known 3-Cl-4-Me-catechol (Gess et al 1971. Tappi 54:1114-1131) is be doubly MOM protected and regioselectively metalated and iodinated to give 12a, then subjected to Heck coupling with 11 to afford 13a.

The same strategy is used to synthesize 3,4-DHSA (14b, FIG. 3A) and other fluorinated and chlorinated derivates (e.g., 1-F—, 1-Cl—, and 2-F—).

### Example 6

### Synthetic Methods for HsaD Inhibitors

Catechol 6 was enzymatically transformed for HsaD studies (FIG. 10). 3,4-DHSA (14b) will be prepared analogously to 2-Cl-3,4-DHSA (14a, FIG. 23). 4,9-DHSAs and 5-Me-10-Cl-HOPODAs were produced from the corresponding 3,4-DHSAs and 2,3-DHDS using HsaC, similar to the production of HOPDAs from DHB, and extracted and HPLC purified as described (Horsman et al 2006. Biochemistry 45:11071-11086)

### Example 7

### Synthetic Methods for HsaA/B Inhibitors

Synthetic methods for inhibitors of HsaAB are shown in FIG. 7. 4-F-3-HSA, is synthesized from 2-F-3-Br-4-Me-MOM phenol. In another example, 7,7-dihydroxymethyl-3-HSA, is synthesized from commercially available 3-Br-4-Me phenol (Aldrich Chemical Co.).

### Example 8

### Synthetic Methods for KstD, KshA/B Inhibitors

Synthetic methods for inhibitors of KstD (2-F-4-AD) and KshAB (2-C; -4-AD) are shown in FIGS. 5 and 6. 2-F- and 2-Cl 4-AD (5S, 8R, 9S, 10R, 13S, 14S; or isomer 5S, 8R, 9S, 10R, 13S, 14S; or racemic mixture thereof) is synthesized from testosterone and 17-hydroxy-(5α)-androstan-3-one (5S, 8R, 9S, 10S, 13S, 14S, 17S; or isomer 5R, 8R, 9S, 10S, 13S, 14S, 17S; or racemic mixture thereof), respectively (FIGS. 5, 6). 2-F-4-AD (19) is prepared by classical fluorination (Nathan et al 1959. J Organic Chemistry) and modified Swern oxidation (Nishide et al 2004. Monatshefte fuer Chemie 135:189-200) starting with the inexpensive testosterone (17). Similarly, 2-Cl-4-AD (24) is obtained starting from commercially available 17-hydroxy-(5α)-androstan-3-one (20) using described methods (Nishide, supra; Velluz et al 1953. Bulletin de la Societe Chimique de France 905; Carrington et al 1961. J. Chem Soc 4560-72).

### Example 9

### Inhibition of M. tuberculosis HsaC

3-Chlorocatechol (3-CC) and 2′,6′-diCl DHB were assayed for inhibition of cloned M tuberculosis HsaC using the spectroscopic assay described by VAILLAINCOURT 1998, supra. Inhibition by each of 2′,6′-diCl DHB and 3-CC was evaluated as described by DAI et al 2002. Nature Structural Biology 9:934-939 and Vaillancourt et al. 2002, J. Biol. Chem., 277, 2019-2027, respectively. The substrate analog (3CC) was found to inhibit HsaC with an IC₅₀ of 1 mM. 3CC also inactivated HsaC in a manner consistent with oxidation of the active site Fe(II). The substrate analogue 2′,6′-diCl DHB was found to inhibit HsaC with an IC₅₀ of 0.1 mM.

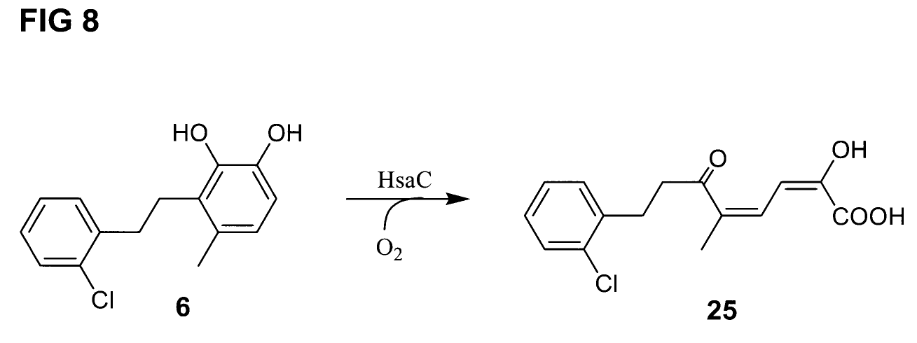

FIG. 8 shows a mass spectrum of TMS-derivatized 3-HSA. The molecular ion (m/z=372) corresponds to derivatized 3-HSA (structure shown in inset). The ion at m/z=206 results from fragmentation between carbons 7 and 8 (dashed red line in the structure). The intensities of the ions were adjusted relative to that of the TMS peak at m/z=73. The cleaved fragment (372−206=166) is the same as we observed in 3,4-DHSA [11].

All citations are herein incorporated by reference.

One or more currently preferred embodiments have been described by way of example. It will be apparent to persons skilled in the art that a number of variations and modifications can be made without departing from the scope of the invention as defined in the claims.

1.-12. (canceled)

13. A method of treating tuberculosis in a subject in need thereof, the method comprising administering an inhibitor of a KshAB complex molecule, a KstD molecule, a HsaAB complex molecule, a HsaC molecule or a HsaD molecule to said subject.

14. The method of claim 13 wherein the inhibitor of the KshAB complex molecule is selected from one or more of the group consisting of 2-chloro-4-androstene-3,17-dione α enantiomer, 2-chloro-4-androstene-3,17-dione β enantiomer, 1-nitro-4-androstene-3,17-dione (1-nitro-4-AD) α enantiomer, 1-nitro-4-androstene-3,17-dione (1-nitro-4-AD) β enantiomer, 4-fluoro-4-androstene-3,17-dione α enantiomer, and 4-fluoro-4-androstene-3,17-dione β enantiomer.

15. The method of claim 13 wherein the KshAB complex molecule comprises a KshA polypeptide comprising an amino acid sequence substantially identical to the amino acid sequence set forth in SEQ ID NO: 17 and a KshB polypeptide comprising an amino acid sequence substantially identical to the amino acid sequence set forth in SEQ ID NO: 19.

16. The method of claim 13 wherein the inhibitor of the HsaAB complex molecule is selected from one or more of the group consisting of 2-chloro-3-hydroxy-9,10-seconandrost-1,3,5(10)-triene-9,17-dione, 1-nitro-10-chloro-3-hydroxy-6-methyl-7,8-dihydrostilbene, 4-fluoro-3-hydroxy-9,10-seconandrost-1,3,5(10)-triene-9,17-dione and 7-hydroxymethyl-7-hydroxyethyl-10-Cl-3-hydroxy-6-methyl-7,8-dihydrostilbene.

17. The method of claim 13 wherein the HsaAB complex comprises a HsaA polypeptide comprising an amino acid sequence substantially identical to the amino acid sequence set forth in SEQ ID NO: 21 and a HsaB polypeptide comprising an amino acid sequence substantially identical to the amino acid sequence set forth in SEQ ID NO: 23.

18. The method of claim 13 wherein the inhibitor of the HsaC molecule is selected from one or more of the group consisting of 2-chloro-3,4-dihydroxy-9,10-seconandrost-1,3,5(10)-triene-9,17-dione, 1-nitro-2,3-dihydroxy-6-methyl-7,8-dihydrostilbene, 7,7-dihydroxymethyl-3,4-dihydroxy-9,10-seconandrost-1,3,5(10)-triene-9,17-dione, 7-hydroxymethyl-7-hydroxyethyl-10-Cl-2,3-dihydroxy-6-methyl-7,8-dihydrostilbene, 3-chlorocatechol and 2′,6′-diCl-2,3-dihydroxybiphenyl.

19. The method of claim 13 wherein the HsaC molecule comprises a polypeptide comprising an amino acid sequence substantially identical to the amino acid sequence set forth in SEQ ID NO: 25.

20. The method of claim 13 wherein the inhibitor of the HsaD molecule is 3-chloro-4,5-9,10-diseco-3-hydroxy-5,9,17-trioxoandrosta-1(10),2-diene-4-oic acid.

21. The method of claim 13 wherein the HsaD molecule comprises a polypeptide comprising an amino acid sequence substantially identical to the amino acid sequence set forth in SEQ ID NO: 27.

22. The method of claim 13 wherein the subject is a human.

23. The method of claim 13 wherein the subject is a guinea pig.

24. The method of claim 13 wherein the subject is a mouse.

25. A compound selected from Formula I: 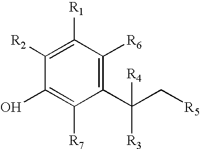
wherein R₁, R₂, R₆ are independently selected from the group consisting of Cl, F, Br, I, —NO₂, —CH═O, —CR═O, —COOH, COOR, CONR₂, COCl, and —CX₃, where X is selected from the group consisting of Cl, Br, F, I, —CN, —SO₃H, —NH₃⁺ and NR₃⁺;
R₇ is any substituent, from one to 10 atoms;
R₃ and R₄ are independently selected from the group consisting of H, —CH₂OH, —CH₂CH₂OH, —CH₂Cl, —CH₂CH₂Cl, —CH₂NH₂, or —CH₂CCl₃, and
R₅ is selected from the group consisting of cycloalkanone, phenyl, purine, pyrimidine and bicycloalkanone; 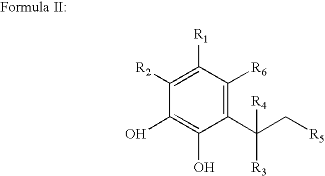
wherein R₁, R₂, R₆ are independently selected from the group consisting of Cl, F, Br, I, —NO₂, —CH═O, —CR═O, —COOH, COOR, CONR₂, COCl, and —CX₃, where X is selected from the group consisting of Cl, Br, F, I, —CN, —SO₃H, —NH₃⁺ and NR₃⁺;
R₃ and R₄ are independently selected from the group consisting of H, —CH₂OH, —CH₂CH₂OH, —CH₂Cl, —CH₂CH₂Cl, —CH₂NH₂, and —CH₂CCl₃; and
R₅ is selected from the group consisting of cycloalkanone, phenyl, purine, pyrimidine, bicycloalkanone and heterocycle; or 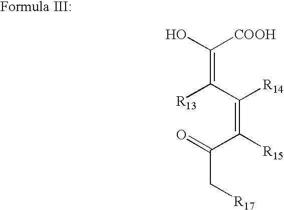
wherein R₁₃, R₁₄ and R₁₅ are independently selected from the group consisting of Cl, F, Br, I, —NO₂, —CH═O, —CR═O, —COOH, COOR, CONR₂, COCl, —CX₃, H, —CH₂OH, —CH₂CH₂OH, —CH₂Cl, —CH₂CH₃, —CH₂ CH₂Cl, —CH₂NH₂, or —CH₂CCl₃; where X is a halogen (Cl, Br, F, I), —CN, —SO₃H, —NH₃⁺, NR₃⁺; and
R₁₇ is selected from the group consisting of Cl, F, Br, I, cycloalkanone, phenyl, purine, pyrimidine, bicycloalkanone, heterocycle, cycloalkanone comprising a halogen substituent group, phenyl comprising a halogen substituent group, purine comprising a halogen substituent group, pyrimidine comprising a halogen substituent group, bicycloalkanone comprising a halogen substituent group and heterocycle comprising a halogen substituent group.

26. The compound of claim 25, wherein the compound of Formula I is selected from one or more of the group consisting of: 2-chloro-3-hydroxy-9,10-seconandrost-1,3,5(10)-triene-9,17-dione, 1-nitro-10-chloro-3-hydroxy-6-methyl-7,8-dihydrostilbene, 4-fluoro-3-hydroxy-9,10-seconandrost-1,3,5(10)-triene-9,17-dione, and 7-hydroxymethyl-7-hydroxyethyl-10-Cl-3-hydroxy-6-methyl-7,8-dihydrostilbene.

27. (canceled)

28. The compound of claim 25, wherein the compound of Formula II is selected from one or more of the group consisting of 2-chloro-3,4-dihydroxy-9,10-seconandrost-1,3,5(10)-triene-9,17-dione, 1-nitro-2,3-dihydroxy-6-methyl-7,8-dihydrostilbene, 7,7-dihydroxymethyl-3,4-dihydroxy-9,10-seconandrost-1,3,5(10)-triene-9,17-dione, 7-hydroxymethyl-7-hydroxyethyl-10-Cl-2,3-dihydroxy-6-methyl-7,8-dihydrostilbene, 3-chlorocatechol and 2′,6′-diCl-2,3-dihydroxybiphenyl.

29. (canceled)

30. The compound of claim 25, wherein the compound of Formula III is selected from one or more of the group consisting of 3-chloro-4,5-9,10-diseco-3-hydroxy-5,9,17-trioxoandrosta-1(10),2-diene-4-oic acid(3-Cl-4,9-DSHA) and (2E,4Z)-8-(2-chlorophenyl)-2-hydroxy-4,5-dimethyl-6-oxoocta-2,4-dienoic acid (4,5-diMe-10-Cl-HOPODA).

31. A pharmaceutical composition comprising a compound according to claim 25, and a pharmaceutically acceptable carrier.

32. The pharmaceutical composition of claim 31 further comprising a therapeutic compound for treating tuberculosis.

33. The pharmaceutical composition of claim 32 wherein said therapeutic compound is selected from one or more of the group consisting of isoniazid (INH), rifampin (RIF), pyrazinamide and ethambutol.

34. A kit comprising the pharmaceutical composition of claim 31, together with instructions for treating tuberculosis.

35. A method for identifying an inhibitor of a KshAB complex molecule, a KstD molecule, a HsaAB complex molecule, a HsaC molecule or a HsaD molecule, the method comprising:
a) providing a test compound;
b) providing a KshAB complex molecule, a KstD molecule, a HsaAB complex molecule, a HsaC molecule or a HsaD molecule;
c) contacting the KshAB complex molecule, KstD molecule, HsaAB complex molecule, HsaC molecule or HsaD molecule with the test compound under conditions suitable for activity of the KshAB complex molecule, KstD molecule, HsaAB complex molecule, HsaC molecule or HsaD molecule; and
d) determining the activity of the KshAB complex molecule, KstD molecule, HsaAB complex molecule, HsaC molecule or HsaD molecule in the presence and absence of the test compound;
wherein the test compound is an inhibitor of the KshAB complex molecule, KstD molecule, HsaAB complex molecule, HsaC molecule or HsaD molecule if the activity of the KshAB complex molecule, KstD molecule, HsaAB complex molecule, HsaC molecule or HsaD molecule is greater in the absence of the test compound compared to the presence of the test compound.

36. A method for identifying a modulator of a KshAB complex molecule, a KstD molecule, a HsaAB complex molecule, a HsaC molecule or a HsaD molecule, the method comprising:
a) providing a test compound;
b) providing a reference compound;
c) providing a KshAB complex molecule, a KstD molecule, a HsaAB complex molecule, a HsaC molecule or a HsaD molecule;
c) contacting the KshAB complex molecule, KstD molecule, HsaAB complex molecule, HsaC molecule or HsaD molecule with the test compound and with the reference compound under conditions suitable for activity of the KshAB complex molecule, KstD molecule, HsaAB complex molecule, HsaC molecule or HsaD molecule; and
d) determining the activity of the KshAB complex molecule, KstD molecule, HsaAB complex molecule, HsaC molecule or HsaD molecule in the presence of the test compound and the reference compound;
wherein the test compound is an modulator of the KshAB complex molecule, KstD molecule, HsaAB complex molecule, HsaC molecule or HsaD molecule if the activity of the KshAB complex molecule, KstD molecule, HsaAB complex molecule, HsaC molecule or HsaD molecule is greater or less in the presence of the test compound compared to the presence of the reference compound.


SEQUENCE LISTING (27 sequences)

SEQ ID NO: 1 - DNA, 26 bp, Artificial sequence
  primer or probe
cgactagcatatgagcatccggtcgc

SEQ ID NO: 2 - DNA, 26 bp, Artificial sequence
  primer or probe
cgggatccctgagccgacatcgtttg

SEQ ID NO: 3 - DNA, 31 bp, Artificial sequence
  primer or probe
cgacgtacatatgacagctaccgaggaattg

SEQ ID NO: 4 - DNA, 26 bp, Artificial sequence
  primer or probe
caggatcctcatctgccacctcccag

SEQ ID NO: 5 - DNA, 59 bp, Artificial sequence
  primer or probe
tctggccaccgatttccttctggacgggggtaagtgatgattccggggatccgtcgacc

SEQ ID NO: 6 - DNA, 58 bp, Artificial sequence
  primer or probe
cctcggccgcgacggccccgtccccggtcacctcactcatgtaggctggagctgcttc

SEQ ID NO: 7 - DNA, 21 bp, Artificial sequence
  hsaCR-for3
tccgcaagaaggtcaagatgt

SEQ ID NO: 8 - DNA, 24 bp, Artificial sequence
  hsaCR-rev3
tcttcatgtagaacgacagcatca

SEQ ID NO: 9 - DNA, 14 bp, Artificial sequence
  hsaCR-TP
ccgtcacgtcaacg

SEQ ID NO: 10 - DNA, 21 bp, Artificial sequence
  kshAB-for
tcctcatcgaagggtccaact

SEQ ID NO: 11 - DNA, 24 bp, Artificial sequence
  kshAB-rev
ggatgtagaagaagtgcgccatat

SEQ ID NO: 12 - DNA, 16 bp, Artificial sequence
  kshAB-tp
catcatcgacaacgtc

SEQ ID NO: 13 - DNA, 20 bp, Artificial sequence
  sigAB-for
ttcgcgcctacctcaaacag

SEQ ID NO: 14 - DNA, 18 bp, Artificial sequence
  sigAB-rev
tcagctgcgtggcgtaca

SEQ ID NO: 15 - DNA, 15 bp, Artificial sequence
  sigAB-TP
tagcgctgctcaacg

SEQ ID NO: 16 - DNA, 1161 bp, Artificial sequence
  ksha nucleotide
gtgagtaccgacacgagtggggtcggtgttcgggagatcgatgccggcgccttgccgaccaggtatgcgcgtggctggcattgcctgggcgtcgcgaaggactatttggaagggaagccacacggggtagaggcgttcggcaccaagctggttgtgttcgctgattcccacggggacctgaaagtcctcgacggctactgccggcacatgggcggcgacctgtccgagggcaccgtcaaaggcgacgaggtcgcttgcccgttccacgactggcgctggggtggcgacggccgctgcaaattggtgccgtatgccaggcgcacacccagaatggcgcgcactcggtcgtggacgaccgatgtgcgcagcgggctgctgtttgtctggcacgaccatgagggcaatccacccgaccccgcggtccggatccccgagattcccgaggcggccagcgacgagtggaccgactggcggtggaaccgcatcctcatcgaagggtccaactgccgcgacatcatcgacaacgtcaccgatatggcgcacttcttctacatccacttcggtttgccgacgtacttcaagaacgtcttcgagggccacatcgcctcgcagtatctgcacaacgtgggccggcccgatgtcgacgatctggggacgtcttacggtgaggcgcatctggattcggaggcgtcttacttcgggccgtcgttcatgatcaactggctgcacaaccgctacggcaactacaagtccgagtcgatcctgatcaactgccactacccggtgacccagaactcgttcgtcctgcaatggggcgtcatcgtcgaaaagcccaagggtatgagcgaagagatgaccgacaagttgtcgcgggtgttcaccgagggcgtcagcaagggcttcttgcaggatgtcgagatctggaagcacaagacccgcatcgacaacccgctgctggttgaagaggacggcgccgtgtatcagctgcgccgctggtatgaacagttttatgtcgacgtagccgacataaaaccagagatggtggagcgcttcgagatcgaggtcgacaccaagcgcgccaacgagttctggaatgccgaggtagagaagaacttgaaatcgagagaagtttccgacgacgtgcccgccgagcaacactga

SEQ ID NO: 17 - protein, 386 aa, Artificial sequence
  kshA amino acid
VSTDTSGVGVREIDAGALPTRYARGWHCLGVAKDYLEGKPHGVEAFGTKLVVFADSHGDLKVLDGYCRHMGGDLSEGTVKGDEVACPFHDWRWGGDGRCKLVPYARRTPRMARTRSWTTDVRSGLLFVWHDHEGNPPDPAVRIPEIPEAASDEWTDWRWNRILIEGSNCRDIIDNVTDMAHFFYIHFGLPTYFKNVFEGHIASQYLHNVGRPDVDDLGTSYGEAHLDSEASYFGPSFMINWLHNRYGNYKSESILINCHYPVTQNSFVLQWGVIVEKPKGMSEEMTDKLSRVFTEGVSKGFLQDVEIWKHKTRIDNPLLVEEDGAVYQLRRWYEQFYVDVADIKPEMVERFEIEVDTKRANEFWNAEVEKNLKSREVSDDVPAEQH

SEQ ID NO: 18 - DNA, 1077 bp, Artificial sequence
  kshB nucleotide
ttgaccgaggcaattggagacgagccactcggcgaccacgtccttgaactgcagatcgccgaggtcgtcgacgaaaccgacgaggcgcgatcgctggtcttcgcggtgcccgacggatcggacgacccggagatcccccctcggcgcctgcgttacgcccccggccaattcttgacgctgcgcgtgcccagcgagcgtaccggttcggtggcgcgctgctactcgttgtgcagttcgccctacaccgacgacgccttggcggtcacggtcaaacgaaccgccgacgggtacgcctccaactggttgtgcgatcacgcgcaggtgggcatgcgcatccacgtgctggccccgtcgggcaacttcgtccccacaaccctcgacgccgatttcctcctgctggcagcgggtagcggcatcaccccgatcatgtcgatctgcaaatcggcgcttgccgagggcggtggacaggtgacgctgctctacgccaaccgcgacgaccgctcggtcatcttcggagacgcgctgcgcgagttggcggcgaagtatcccgaccggctcacggtgctgcactggctagagtcgctgcaggggctgccgagcgcgagcgcgctggccaagctcgtcgcgccctacaccgaccggccggtgttcatctgtgggcccggcccgttcatgcaggcggcccgggacgccctggcggcgctgaaagtgcccgcccaacaggtgcacatcgaggtgttcaagtcgctggaatcggatccgttcgcggccgtcaaggtcgacgacagcggtgacgaggcgccggcgaccgcggtggtggaactcgacggccaaacccacaccgtctcctggccgcgcaccgccaagctgctcgacgtgctgctggccgcgggcctggacgcgccgttctcctgccgggaaggccactgcggtgcgtgtgcgtgcaccctgcgcgccggcaaagtgaatatgggagtcaacgacgtgctcgagcagcaggatctcgatgagggactgattttggcctgtcaatctcgcccggaatctgattcggtggaagtgacctacgacgagtag

SEQ ID NO: 19 - protein, 358 aa, Artificial sequence
  kshB amino acid
LTEAIGDEPLGDHVLELQIAEVVDETDEARSLVFAVPDGSDDPEIPPRRLRYAPGQFLTLRVPSERTGSVARCYSLCSSPYTDDALAVTVKRTADGYASNWLCDHAQVGMRIHVLAPSGNFVPTTLDADFLLLAAGSGITPIMSICKSALAEGGGQVTLLYANRDDRSVIFGDALRELAAKYPDRLTVLHWLESLQGLPSASALAKLVAPYTDRPVFICGPGPFMQAARDALAALKVPAQQVHIEVFKSLESDPFAAVKVDDSGDEAPATAVVELDGQTHTVSWPRTAKLLDVLLAAGLDAPFSCREGHCGACACTLRAGKVNMGVNDVLEQQDLDEGLILACQSRPESDSVEVTYDE

SEQ ID NO: 20 - DNA, 1185 bp, Artificial sequence
  hsaA nucleotide
gtgacgtccattcaacagcgtgatgctcagtctgtcttggctgccatcgataatctgctgccggagattcgggatcgcgcccaggccacggaggatctgcggcggctgcccgacgagaccgtcaaggcgctcgacgacgtcggcttcttcaccctgttgcagccgcagcagtggggcggactgcagtgcgaccctgcgctgttcttcgaggcgacgcgtcgactggcgagcgtgtgtggttccaccgggtgggtgagctcaatcgtcggcgtgcacaactggcatctggcgctgttcgaccaacgggcccaagaggaggtctggggcgaggacccgagtacgcggatctcgtcctcgtacgcaccgatgggggcgggcgtggtggtcgacggtggctacctggtcaacgggtcgtggaactggtcctcgggctgcgaccatgccagctggacgttcgtcggcggcccggtcatcaaggacggccggccggtggacttcggcagtttcctgatcccgcgcagcgaatacgagatcaaggacgtgtggtacgtggtcggcttacgtggtaccggcagcaacaccctggtggtcaaggatgtctttgtgccccggcaccggttcttgtcgtacaaggcgatgaacgaccacaccgcgggagggctggcgaccaacagcgcacctgtctataaaatgccatggggcacaatgcatcccactacgatttcggcgccgattgtcggcatggcctacggtgcctacgccgcgcacgtcgagcaccagggcaagcgggtgcgcgcggcgttcgccggggaaaaggccaaagacgacccgtttgccaaggtccgtatcgccgaggcggccagcgacatcgacgccgcgtggcgccaactgatcggtaatgtgagcgacgaatacgcgctgctggctgccggcaaggagattccgttcgaactgcgcgcccgtgcgcgccgcgaccaggtgcgcgccaccggtcgctcaatcgcctcgatcgaccggctgttcgaggcgtccggtgccaccgcgttgtccaatgaggctccgattcaacggttctggcgcgacgcgcacgccggtcgggtgcacgccgccaacgaccccgagcgggcatacgtgatctttggaaaccacgagttcgggttgccgcccggcgacaccatggtctag

SEQ ID NO: 21 - protein, 394 aa, Artificial sequence
  hsaA amino acid
VTSIQQRDAQSVLAAIDNLLPEIRDRAQATEDLRRLPDETVKALDDVGFFTLLQPQQWGGLQCDPALFFEATRRLASVCGSTGWVSSIVGVHNWHLALFDQRAQEEVWGEDPSTRISSSYAPMGAGVVVDGGYLVNGSWNWSSGCDHASWTFVGGPVIKDGRPVDFGSFLIPRSEYEIKDVWYVVGLRGTGSNTLVVKDVFVPRHRFLSYKAMNDHTAGGLATNSAPVYKMPWGTMHPTTISAPIVGMAYGAYAAHVEHQGKRVRAAFAGEKAKDDPFAKVRIAEAASDIDAAWRQLIGNVSDEYALLAAGKEIPFELRARARRDQVRATGRSIASIDRLFEASGATALSNEAPIQRFWRDAHAGRVHAANDPERAYVIFGNHEFGLPPGDTMV

SEQ ID NO: 22 - DNA, 564 bp, Artificial sequence
  hsaB nucleotide
atgtcggctcagatcgatccacgcacgttccgctccgtgctcggtcagttctgtacggggatcactgtcatcaccactgtgcacgacgacgtgcccgtcgggtttgcgtgccagtcgttcgcggcgctgtcgttggaaccgccgttggtgctgttctgccccactaaggtgtcgcggtcctggcaggccatcgaggccagcggccggttctgcgtcaacgtactgaccgaaaagcagaaagacgtctcggcgcgattcggctcgaaggagccggacaagttcgccggtatcgattggcgcccttcggaactaggctcaccgatcatcgagggatcgctggcctacattgactgcacggtggcgtcggtgcacgacggcggcgatcacttcgtggtgttcggtgccgtcgagtcactgtcagaggtcccggcggtcaagccgcggccgctgctgttctatcgcggcgactacaccggcatcgagccggaaaagaccacgccggcgcactggcgcgacgacctggaggctttcctcatcaccaccacccaggacacctggctctag

SEQ ID NO: 23 - protein, 187 aa, Artificial sequence
  hsaB amino acid
MSAQIDPRTFRSVLGQFCTGITVITTVHDDVPVGFACQSFAALSLEPPLVLFCPTKVSRSWQAIEASGRFCVNVLTEKQKDVSARFGSKEPDKFAGIDWRPSELGSPIIEGSLAYIDCTVASVHDGGDHFVVFGAVESLSEVPAVKPRPLLFYRGDYTGIEPEKTTPAHWRDDLEAFLITTTQDTWL

SEQ ID NO: 24 - DNA, 903 bp, Artificial sequence
  hsaC nucleotide
atgagcatccggtcgctgggctatctgcgcatcgaggccactgacatggctgcctggcgggagtatggactgaaggtcctgggcatggtcgagggcaaaggggccccagaaggggccctgtatctgcggatggatgactttccggcccggctggtggtcgtgcccggtgagcacgacaggctgttggaggccggttgggaatgcgcgaatgccgaaggcctgcaggaaatccggaaccgcctggatttggagggcacgccgtacaaagaggccaccgccgccgagctggccgatcgccgggtggacgagatgatccggtttgccgacccttccggcaattgcctggaggtcttccacggtaccgcgctggaacaccgccgagtggtcagcccgtatgggcacaggttcgtcaccggtgagcagggtatgggccatgtggtgctgtccacccgcgacgatgctgaggcgttgcatttctaccgagacgtgctcggctttcggctgcgtgactcgatgcggctaccgccacagatggtagggcggcccgccgacgggccgccagcctggctgcgtttcttcggctgtaacccgcgtcaccactcgttggccttcctgccgatgccgacgtccagcggcatcgtgcacctgatggtcgaggtcgagcaggccgacgacgtgggcctgtgcctggaccgggcgttgcggcgcaaggtgccgatgtcggccaccctgggccggcatgtcaacgacctgatgctgtcgttctacatgaagacgcccggtggcttcgacatcgaattcggttgtgagggcaggcaagtcgacgatcgggactggatcgcccgggagagtactgcggtcagcctatggggtcacgatttcaccgtaggcgctcgcggctag

SEQ ID NO: 25 - protein, 300 aa, Artificial sequence
  hsaC amino acid
MSIRSLGYLRIEATDMAAWREYGLKVLGMVEGKGAPEGALYLRMDDFPARLVVVPGEHDRLLEAGWECANAEGLQEIRNRLDLEGTPYKEATAAELADRRVDEMIRFADPSGNCLEVFHGTALEHRRVVSPYGHRFVTGEQGMGHVVLSTRDDAEALHFYRDVLGFRLRDSMRLPPQMVGRPADGPPAWLRFFGCNPRHHSLAFLPMPTSSGIVHLMVEVEQADDVGLCLDRALRRKVPMSATLGRHVNDLMLSFYMKTPGGFDIEFGCEGRQVDDRDWIARESTAVSLWGHDFTVGARG

SEQ ID NO: 26 - DNA, 876 bp, Artificial sequence
  hsaD nucleotide
atgacagctaccgaggaattgacgttcgaatccacctcgcgctttgcggaagtggacgtcgacgggccgctgaaactgcactaccacgaggccggcgtgggcaacgaccagacggtggtgctactgcacggcggtgggcccggcgcggcgagctggacgaacttctcgcgtaatatcgcggtgctggcgcggcactttcatgtgctggccgtcgaccagcccggttacggccattccgacaagcgggccgagcacggccagttcaatcgctatgccgcgatggcgctgaaggggctcttcgatcagctggggctggggcgggtaccgctggtgggcaactcgttgggcgggggaaccgcggtccggtttgcgctggactacccggcccgggcaggacggttagtgctgatgggcccggggggcctgagtatcaacctgtttgcgcccgacccgaccgagggagtcaaacggctgtcgaagttctccgttgcgcccacccgggagaacctcgaggcgttcctgcgggtcatggtctacgacaagaacctgatcacccccgagttggtggatcagcggtttgcgctggccagcaccccggagtcgttgacggcaacacgggcgatgggaaagtcgttcgccggagccgacttcgaggccggcatgatgtggcgcgaggtgtatcggctgcgccagccggtgttgctgatctggggtcgtgaggaccgggtcaacccgctggacggcgcgctggttgcgttgaaaacgattccgcgtgcgcagctgcacgtattcgggcagtgtgggcattgggtgcaggtggagaagttcgacgagttcaacaagctgacgattgaatttctgggaggtggcagatga

SEQ ID NO: 27 - protein, 291 aa, Artificial sequence
  hsaD amino acid
MTATEELTFESTSRFAEVDVDGPLKLHYHEAGVGNDQTVVLLHGGGPGAASWTNFSRNIAVLARHFHVLAVDQPGYGHSDKRAEHGQFNRYAAMALKGLFDQLGLGRVPLVGNSLGGGTAVRFALDYPARAGRLVLMGPGGLSINLFAPDPTEGVKRLSKFSVAPTRENLEAFLRVMVYDKNLITPELVDQRFALASTPESLTATRAMGKSFAGADFEAGMMWREVYRLRQPVLLIWGREDRVNPLDGALVALKTIPRAQLHVFGQCGHWVQVEKFDEFNKLTIEFLGGGR